(19) United States
(12) Patent Application Publication
(10) Pub. No.: US 2010/0047273 A1
(43) Pub. Date: Feb. 25, 2010

(54) Coxsackie B virus and type 1 diabetes

(76) Inventors: Rino Rappuoli, Siena (IT); Francesco Dotta, Roma (IT); Piero Marchetti, Pisa (IT)

Correspondence Address: NOVARTIS VACCINES AND DIAGNOSTICS INC., INTELLECTUAL PROPERTY- X100B, P.O. BOX 8097, Emeryville, CA, 94662-8097, US

(21) Appl. No.: 12/151,725

(22) Filed: May 7, 2008

Related U.S. Application Data
(60) Provisional application No. 60/928,133, filed on May 7, 2007.

(51) Int. Cl. A61K 39/12; C12Q 1/70; C07H 21/00; C07K 7/06; C07K 14/00; C07K 16/00; A61P 37/04; A61P 31/12

(52) U.S. Cl. 424/204.1; 435/5; 536/23.1; 530/329; 530/350; 530/387.1

(57) ABSTRACT

Type 1 diabetes mellitus is characterized by loss of pancreatic insulin-producing beta cells, resulting in insulin deficiency. The usual cause of this beta cell loss is autoimmune destruction. Coxsackie virus has been detected in human pancreatic beta cells and causes insulitis. This non-destructive islet inflammation does not itself cause diabetes, but this disease will occur if viral infection is followed by a separate autoimmune response. The insulitis is mediated mainly by natural killer cells. Islets from coxsackie virus positive samples displayed reduced insulin secretion in response to glucose and other secretagogues. Virus extracted from positive islets was able to infect beta cells from human islets of non-diabetic donors, causing viral inclusions and signs of pyknosis.

All publications, patents, patent applications and online information mentioned in this specification are incorporated herein by reference to the same extent as if each individual document were specifically and individually indicated to be incorporated by reference.

### TECHNICAL FIELD

The present invention relates to the involvement of viruses in type 1 diabetes, and it is an object of the invention to provide further and improved materials and methods that can be used in the diagnosis, prevention and treatment of type 1 diabetes.

### BACKGROUND ART

Type 1 diabetes mellitus (previously known as IDDM) is characterized by loss of pancreatic insulin-producing beta cells, resulting in insulin deficiency. The usual cause of this beta cell loss is autoimmune destruction.

It has been proposed that the autoimmune destruction may be linked to a viral infection. For a virus to act as a trigger for autoimmune beta cell destruction, various mechanisms have been proposed. For instance, cytolytic infection of beta cells could occur, leading to their destruction and/or to the release of normally-sequestered antigens, which might then trigger pathogenic autoreactive T-cell responses. Alternatively, epitopes displayed by the virus may elicit auto-reactive antibodies and/or T cells, thereby providing the basis of autoimmunity.

Various viruses have been linked to type 1 diabetes [1]. For instance, reference 2 noted in 2001 that 13 different viruses had been reported to be associated with its development in humans and in various animal models, including mumps virus, rubella virus, cytomegalovirus and coxsackie B virus (CBV). In 2004, however, a systematic review of published case-control studies [3] concluded that there was no convincing evidence for or against an association between CBV infection and type 1 diabetes.

### DISCLOSURE OF THE INVENTION

Whereas prior art associations between CBV and type 1 diabetes have been based on epidemiological studies, correlations, animal models or in vitro infection studies, the inventors have for the first time provided direct evidence of a link by detecting virus in human pancreatic beta cells. This finding provides various therapeutic, prophylactic and diagnostic opportunities.

Moreover, the findings contradict previous suggestions that CBV infection is a direct trigger of diabetes-causing autoimmunity. Rather, infection is associated with non-destructive islet inflammation (insulitis), such that beta cells survive infection but their insulin secretion is inhibited. Destruction of the cells occurs only when viral infection is followed by a separate autoimmune response. The separation of infection and diabetes again offers therapeutic, prophylactic and prognostic opportunities.

Furthermore, post-infection insulitis is mediated mainly by natural killer cells. Inhibition of NK cells may thus have therapeutic potential in infected patients.

The invention is based on work performed in Italy with a new “Tuscany” strain of coxsackie B4 virus, whose genome sequence is SEQ ID NO: 1 herein. The invention is not restricted to this particular strain, however, and can be applied more generally e.g. to any coxsackie virus, in particular a coxsackie B virus, including any coxsackie B4 virus.

The invention provides a method for preventing or treating type 1 diabetes in a patient, comprising a step of administering to the patient an antiviral compound effective against a coxsackie virus.

The invention also provides a method for preventing or treating type 1 diabetes in a patient, comprising a step of administering to the patient a composition that comprises a coxsackie virus immunogen.

The invention also provides a method for preventing or treating type 1 diabetes in a patient, comprising an immunomodulatory compound effective to inhibit natural killer cell activity.

The invention also provides an assay method comprising a step of detecting in a patient sample the presence or absence of a coxsackie virus or an expression product thereof.

The invention also provides an assay method comprising a step of detecting in a patient sample the presence or absence of an immune response against a coxsackie virus.

The invention also provides nucleic acids and polypeptides derived from coxsackie B4 virus having genome sequence SEQ ID NO: 1, and materials related thereto.

### Administration of Antiviral Compounds

The invention provides a method for preventing or treating type 1 diabetes in a patient, comprising a step of administering to the patient an antiviral compound effective against a coxsackie virus.

Various antiviral compounds effective against coxsackie viruses are known in the art. For instance: reference 4 reports that pleconaril is active against coxsackie B4 virus; reference 5 reports that C-5 substituted uracil derivatives of 1-ascorbic acid are active against coxsackie B4 virus; reference 6 reports that homoisoflavonoids and substituted homoisoflavonoids are active against various coxsackie B virus types; etc. These and other antivirals may be used.

Further antivirals that may be useful with the invention include, but are not limited to: galangin (3,5,7-trihydroxyflavone); bupleurum kaoi; neopterin; Ardisia chinensis extract; galloyltricetifavans, such as 7-O-galloyltricetifavan and 7,4′-di-O-galloyltricetifavan; purine and pyrimidine cis-substituted cyclohexenyl and cyclohexanyl nucleosides; benzimidazole derivatives; pyridazinyl oxime ethers; enviroxime; disoxaril; arildone; PTU-23; HBB; S-7; 2-(3,4-dichloro-phenoxy)-5-nitrobenzonitrile; 6-bromo-2,3-disubstituted-4(3H)-quinazolinones; 3-methylthio-5-aryl-4-isothiazolecarbonitriles; quassinoids, such as simalikalactone D; 5′-Nor carbocyclic 5′-deoxy-5′-(isobutylthio)adenosine and its 2′,3′-dideoxy-2′,3′-didehydro derivative; oxathiin carboxanilide analogues; vinylacetylene analogs of enviroxime; Dehydroepiandrosterone (5-androsten-3 beta-ol-17-one, DHEA); flavans, isoflavans and isoflavenes substituted with chloro, cyano or amidino groups, such as substituted 3-(2H)-isoflavenes carrying a double bond in the oxygenated ring e.g. 4′-chloro-6-cyanoflavan and 6-chloro-4′-cyanoflavan; 4-diazo-5-alkylsulphonamidopyrazoles; 3′-deoxy-3′-fluoro- and 2′-azido-3′-fluoro-2′,3′-dideoxy-D-ribofuranosides of natural heterocyclic bases; etc.

Mixtures of two or more antivirals may be used. For instance, reference 7 reports that certain combinations may show synergistic activity.

In addition to small organic antivirals, cytokine therapy may be used e.g. with interferons. For example, interferon α (in particular IFN-α2a) has been used to treat CBV infections. Compounds that elicit an interferon α response can also be used e.g. inosine-containing nucleic acids such as ampligen.

Nucleic acid approaches can also be used against CBV, such as antisense [8] or small inhibitory RNAs [9].

### Immunisation

The invention provides a method for preventing or treating type 1 diabetes in a patient, comprising a step of administering to the patient an immunogenic composition. The immunogenic composition includes a coxsackie virus immunogen.

The coxsackie virus immunogen may take various forms. For instance, it may be a live attenuated virus. It may be an inactivated whole virion. It may be a split virion. It may be a purified viral polypeptide (natural or recombinant), such as a polypeptide comprising a VP1, VP2, VP3, VP4, 2A, 2B, 2C, 3A, 3B, 3C or 3D sequence. Virion surface proteins VP1, VP2 and VP3 are particularly useful as immunogens. If VP4 protein is used then it may be myristoylated at the C-terminus.

Neutralising antibody responses have previously been obtained in animal models using a live attenuated B3 virus, a whole virion vaccine inactivated by β-propiolactone, and a purified polypeptide vaccine [10].

As an alternative to delivering polypeptide-based immunogens themselves, nucleic acids encoding the polypeptides may be administered such that, after delivery to the body, the polypeptides are expressed in situ. Nucleic acid immunization against coxsackie B viruses has previously been reported [11-14] for the VP1 polypeptide. Nucleic acid immunization typically utilizes a vector, such as a plasmid, comprising: (i) a promoter; (ii) a sequence encoding the immunogen, operably linked to said promoter; and (iii) a selectable marker. Vectors often further comprise (iv) an origin of replication; and (v) a transcription terminator downstream of and operably linked to (ii). Components (i) & (v) will usually be eukaryotic, whereas (iii) and (iv) are prokaryotic.

An immunogenic composition may additionally comprise an adjuvant. For example, the composition may comprise one or more of the following adjuvants: (1) oil-in-water emulsion formulations, saponins (such as QS21), ISCOMs (immunostimulating complexes), 3-O-deacylated MPL (3dMPL), oligonucleotides comprising CpG motifs i.e. containing at least one -C-G- dinucleotide, aluminium salts including hydroxides and/or phosphates, chitosan, cholera toxin or E. coli heat labile toxin or detoxified mutants thereof, microparticles of poly(α-hydroxy)acids such as PLG, etc.

A polypeptide used in an immunogenic composition may have an amino acid sequence of a natural coxsackievirus polypeptide (precursor or mature form) or it may be artificial e.g. it may be a fusion protein or it may comprise a fragment (e.g. including an epitope) of a natural coxsackievirus sequence.

Useful polypeptides and nucleic acids from the Tuscany strain of CBV4 are described in more detail below.

### NK Modulation

NK cells are a subset of lymphocytes that act as an initial immune defense against tumor cells and virally infected cells. It has been found that these cause insulitis after CBV infection of pancreatic beta cells, and the invention provides a method for preventing or treating type 1 diabetes in a patient, comprising an immunomodulatory compound effective to inhibit natural killer cell activity. In general, however, total inhibition is not desirable.

Compounds effective to inhibit NK function include, but are not limited to: steroids, such as methylprednisolone; tributyltin; Ly49 ligands, such as H-2D(d); soluble HLA-G1; CD94/NKG2A; CD244 ligands; etc.

Compounds may act directly or indirectly on the NK cells. For example, tributyltin acts directly on NK cells. In contrast, CD4+CD25+ T regulatory cells can inhibit NK cells, and so a compound may be administered to a patient in order to promote such CD4+CD25+ T cells and thereby indirectly inhibit NK cells.

### Diagnostic and Prognostic Assays

The invention provides assay methods comprising a step of detecting in a patient sample the presence or absence of (a) a coxsackie virus or an expression product thereof, and/or (b) an immune response against a coxsackie virus. Detection of a presence indicates that the patient has been infected by coxsackie virus and is thus at risk of the downstream diabetes-related consequences. Assays of the invention can therefore be used for diagnosing type 1 diabetes in a patient. They can also be used for diagnosing future diabetes risk or in diabetes prognosis.

It will be appreciated that “diagnosis” can range from a definite clinical diagnosis of disease to an indication that the patient is at risk and so should undergo further testing that may then lead to a definite diagnosis. For example, the method of the invention can be used as part of a screening process, with positive samples being subjected to further analysis. In general, the invention will be used to detect coxsackie virus infection, in particular in relation to pancreatic beta cells, and the presence of infection will be used, alone or in combination with other test results, as the basis of diagnosis or prognosis.

Diagnostic assays of the invention may detect a coxsackie virus (e.g. its single-stranded RNA genome, a provirion, a virion), an expression product of a coxsackie virus (e.g. its anti-genome, a viral mRNA transcript, an encoded polypeptide such as a VP1, VP2, VP3, VP4, 2A, 2B, 2C, 3A, 3B, 3C or 3D), or the product of an immune response against a coxsackie virus (e.g. an antibody against a viral polypeptide, a T cell recognizing a viral polypeptide).

Diagnostic assays for coxsackie viruses are described on pages 758-762 of reference 15, including tests based on viral growth, antibody responses and nucleic acid detection. Moreover, reference 16 discloses primer sets targeting the 5′ UTR, the VP1 region, the 3D region and a long genomic fragment including the 3′ end of VP1, the full length of 2A and 2B, and the 5′ moiety of the 2C-coding region. Reference 17 also discloses various methods for preparing and analyzing coxsackie B4 viruses.

A useful method for detecting RNA is the polymerase chain reaction, and in particular RT-PCR (reverse transcriptase PCR). Further details on nucleic acid amplification methods are given below.

Various techniques are available for detecting the presence or absence of polypeptides in a sample. These are generally immunoassay techniques which are based on the specific interaction between an antibody and an antigenic amino acid sequence in the polypeptide. Suitable techniques include standard immunohistological methods, ELISA, RIA, FIA, immunoprecipitation, immunofluorescence, etc. Sandwich assays are typical. Antibodies against various coxsackie viruses are already commercially available, including ones that can distinguish between different virus groups (e.g. to distinguish B4 from B3) and between different proteins in the same virus (e.g. to distinguish protein VP1 from VP2).

Polypeptides can also be detected by functional assays e.g. assays to detect binding activity or enzymatic activity. Another way of detecting polypeptides of the invention is to use standard proteomics techniques e.g. purify or separate polypeptides and then use peptide sequencing. For example, polypeptides can be separated using 2D-PAGE and polypeptide spots can be sequenced (e.g. by mass spectroscopy) in order to identify if a sequence is present in a target polypeptide. Some of these techniques may require the enrichment of target polypeptides prior to detection; other techniques may be used directly, without the need for such enrichment.

Antibodies raised against a coxsackie virus may be present in a sample and can be detected by conventional immunoassay techniques e.g. using coxsackie virus polypeptides, which will typically be immobilized.

### Prevention and Therapy

The invention can be used to prevent type 1 diabetes in a patient. Such patients will not already be suffering from type 1 diabetes, but they will be at risk of developing type 1 diabetes. In such patients, prevention encompasses both (i) reducing the risk that they will develop type 1 diabetes, and (ii) lengthening the time before they develop type 1 diabetes.

Because it has been found that coxsackie virus infection leads to insulitis, without beta cell destruction, the invention can also be used to treat insulitis in pre-diabetic patients. Such treatment is a further way in which the development and onset of diabetes can be prevented.

The invention can also be used to treat type 1 diabetes in a patient. For instance, therapeutic immunization or antiviral treatment may be used to clear a coxsackie virus infection and then beta cell regeneration can be permitted (optionally in combination with treatment of the autoimmune aspect of type 1 diabetes). The method may be combined with islet transplantation or the transplantation of beta cell precursors or stem cells. The terms “treatment”, “treating”, “treat” and the like refer to obtaining a desired pharmacologic and/or physiologic effect. The effect may be therapeutic in terms of a partial or complete stabilization or cure for type 1 diabetes and/or adverse effect attributable to type 1 diabetes. “Treatment” includes inhibiting a disease symptom (i.e. arresting its development) and relieving the disease symptom, (i.e. causing regression of the disease or symptom).

The invention can be used in conjunction with conventional methods of type 1 diabetes prevention and/or treatment.

The invention can be used with a wide variety of patients, but some embodiments are more useful for specific patient groups. For instance, some embodiments will usually be applied only with patients having a definite coxsackie virus infection, whereas other embodiments may be focused on patients known to be at high risk of developing type 1 diabetes (e.g. with a familial history of the disease, with a HLA-DR3 haplotype and/or a HLA-DR4 haplotype, etc.). For instance, the administration of antiviral compounds will typically be used in pre-diabetic patients having a viral infection, whereas prophylactic immunization will be used more widely (e.g. in high risk groups, or in the population as a whole).

A preferred type of patient for use with diagnostic, prognostic and prophylactic methods of the invention is a patient who has insulitis but has not yet developed type 1 diabetes.

### Patient Samples

Various embodiments of the invention require samples that have been obtained from patients. These samples will generally comprise cells (e.g. pancreatic cells, including beta cells). These may be present in a sample of tissue (e.g. a biopsy), or may be cells which have escaped into circulation. In some embodiments, however, the sample will be cell-free e.g. from a body fluid that may contain coxsackie virions in the absence of patient cells, or a purified cell-free blood sample that may contain anti-viral antibodies.

In general, therefore, the patient sample is tissue sample or a blood sample. Other possible sources of patient samples include isolated cells, whole tissues, or bodily fluids (e.g. blood, plasma, serum, urine, pleural effusions, cerebro-spinal fluid, etc.).

The sample is preferably from a human patient.

Expression products may be detected in the patient sample itself, or may be detected in material derived from the sample (e.g. the lysate of a cell sample, the supernatant of such a cell lysate, a nucleic acid extract of a cell sample, DNA reverse transcribed from a RNA sample, polypeptides translated from a RNA sample, cells derived from culturing cells extracted from a patient, etc.). These derivatives are still “patient samples” within the meaning of the invention.

Detection methods of the invention can be conducted in vitro or in vivo.

In some embodiments of the invention a control may be used, against which coxsackie virus levels in a patient sample can be compared. Analysis of the control sample gives a baseline level against which a patient sample can be compared. A negative control may be a sample from an uninfected patient, or it may be material not derived from a patient e.g. a buffer. A positive control will be a sample with a known level of analyte. Other suitable positive and negative controls will be apparent to the skilled person.

Analyte in the control can be assessed at the same time as in the patient sample. Alternatively, a patient sample can be assessed separately (earlier or later). Rather than actually compare two samples, however, the control may be an absolute value i.e. a level of analyte which has been empirically determined from previous samples (e.g. under standard conditions).

The invention provides an immunoassay method, comprising the step of contacting a patient sample with a polypeptide or antibody of the invention.

### Coxsackie Viruses

The coxsackie viruses are members of the Picornaviridae family, genus Enterovirus. The genome is comprised of single-stranded, positive-sense monopartite RNA. The genome is infectious because it can be translated on entry into a cell and produce all viral proteins required for replication. The 5′ terminus of the viral genome is covalently attached to the VPg viral protein by a O4-(5′-uridyl)-tyrosine linkage. The 3′ terminus has a polyA tail, usually between 35-100 nucleotides long. The coxsackie genome encodes a polyprotein that is eventually cleaved to give 11 proteins (VP1-VP4, 2A-2C and 3A-3D). The VP proteins make up the “P1” region of the genome, encoding viral capsid proteins. The P2 and P3 proteins are involved in protein processing (2A, 3C and 3CD) and genome replication (2B, 2C, 3AB, 3B, 3CD and 3D). The viral lifecycle involves: attachment to a host cell; uncoating and entry; translation; proteolytic processing; synthesis of negative RNA strand; synthesis of positive RNA strands; translation; and virion packaging & assembly. The negative RNA strand has a 5′ polyU sequence, copied from the viral polyA tail.

The viral capsid of coxsackie viruses is made of four structural proteins: VP1, VP2, VP3 and VP4. These four proteins (one copy of each) are arranged in protomers, and the protomers form the virion. VP1, VP2 and VP3 are exposed on the virion surface, whereas VP4 lies on the inner surface. VP4 is often myristoylated at its N-terminus.

Coxsackie viruses are classified into two groups: A and B. Within group A, there are at least 24 antigenic types (type 23 being the same as echovirus 9); within group B there are at least 6 antigenic types. The invention is mainly concerned with coxsackie viruses in group B, and in particular antigenic type 4 i.e. coxsackie B4 viruses. Within the B4 type, at least seven distinct genetic lineages (genotypes) have been circulating in Europe during the period 1959-1998 [17], and the invention can use any of these lineages. The prototype strain of coxsackie B4 virus is “JVB”, originally isolated in New York. The 7395-mer genome of JVB is SEQ ID NO: 15 herein (GenBank X05690; GI:61031), encoding SEQ ID NO: 16. A preferred strain for use with the invention is the Tuscany B4 strain.

### Tuscany Strain of Coxsackie B4 Virus

SEQ ID NO: 1 is the ssRNA genome sequence (omitting its polyA tail) of a specific CBV4 strain isolated in Tuscany, Italy. The 7395-mer genome (SEQ ID NO: 1) encodes a 2183-mer polyprotein (SEQ ID NO: 2) that is cleaved into the 11 mature products SEQ ID NOs: 3 to 13 (see FIG. 1). In its native form, the 5′ terminus of the viral genome is covalently attached to amino acid Tyr-3 of the VPg protein (also known as 3B; SEQ ID NO: 11). The 2C region (SEQ ID NO: 9) encodes the viral RNA helicase. The 2A (SEQ ID NO: 7) and 3C (SEQ ID NO: 12) regions encodes viral proteases, which initially act on the polyprotein as shown in FIG. 2. The 3D region (SEQ ID NO: 13) encodes viral polymerase (a RNA-dependent RNA polymerase).

SEQ ID NO: 14 is a DNA sequence corresponding to the RNA of SEQ ID NO: 1.

FIG. 3 is a dendrogram showing the relationship between SEQ ID NO: 14 and known coxsackie virus genomes. The most closely related sequence is JVB.

In some embodiments of the invention, assays can distinguish between the Tuscany sequence and known prior art sequences i.e. the assays are specific for the Tuscany sequence. To distinguish the Tuscany sequence from the JVB sequence, one or more of the following nucleotides may be tested (numbered according to SEQ ID NO: 1): 136, 137, 171, 546, 812, 1362, 1381, 1385, 2816, 3038, 4034, 4307, 5015, 5117, 5118, 5124, 5176, 5196, 5541, 5687, 5708, 5709, 5710, 5875, 5876, 5939, 6085, 6516, 7385. Similarly, to distinguish from the JVB sequence, one or more of the following amino acids may be tested (numbered according to SEQ ID NO: 2): 207, 213, 214, 1458, 1459, 1461, 1478, 1485, 1656, 1711, 1781, 1925.

An assay of the invention may include a step of checking the nucleotide/amino acid at one of these positions in order to determine whether a particular virus is a Tuscany isolate.

### Nucleic Acids

The invention provides nucleic acid comprising a nucleotide sequence that is a fragment of at least i contiguous nucleotides of SEQ ID NO: 1 or SEQ ID NO: 14. It also provides nucleic acid comprising a nucleotide sequence that has at least a % sequence identity to SEQ ID NO: 1 or SEQ ID NO: 14. It also provides nucleic acid comprising (i) a nucleotide sequence that has at least a % sequence identity to SEQ ID NO: 1 or SEQ ID NO: 14 and (ii) a nucleotide sequence that is a fragment of at least i contiguous nucleotides of SEQ ID NO: 1 or SEQ ID NO: 14.

The invention also provides nucleic acid comprising the complement (including the reverse complement) of such nucleotide sequences. Such nucleic acids may be used e.g. for antisense, for probing, for use as primers, etc.

The percentage value of a is typically at least 50 e.g. 50, 55, 60, 65, 70, 75, 80, 85, 90, 91, 92, 93, 94, 95, 96, 97, 98, 99, 99.5, 99.9 or 100. Nucleic acid sequences which include ‘silent’ changes (i.e. which do not affect the encoded amino acid for a codon) are examples of these nucleic acids.

The value of i is typically at least 6 e.g. 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 60, 70, 80, 90, 100 or more.

The invention also provides nucleic acid of formula 5′-X-Y-Z-3′, wherein: -X- is a nucleotide sequence consisting of x nucleotides; -Z- is a nucleotide sequence consisting of z nucleotides; -Y- is a nucleotide sequence consisting of either (a) a fragment of SEQ ID NO: 1 or SEQ ID NO: 14, or (b) the complement of (a); and said nucleic acid 5′-X-Y-Z-3′ is neither (i) a fragment of SEQ ID NO: 1 or SEQ ID NO: 14 nor (ii) the complement of (i). The -X- and/or -Z- moieties may comprise a promoter sequence (or its complement).

The value of x+z is at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, etc.). The value of x+y+z is usually at least 8 (e.g. at least 9, 10, 11, 12, 13, 14, 15, 16, 17, 18, 19, 20, 21, 22, 23, 24, 25, 30, 35, 40, 45, 50, 60, 70, 80, 90, 100, etc.). In some embodiments, the value of x+y+z is at most 500 (e.g. at most 450, 400, 350, 300, 250, 200, 190, 180, 170, 160, 150, 140, 130, 120, 110, 100, 90, 80, 70, 60, 50, 40, 30, 25, 20, 19, 18, 17, 16, 15, 14, 13, 12, 11, 10, 9, 8).

Preferred fragments of the invention include one or more of the following nucleotides (numbered according to SEQ ID NO: 1): 136, 137, 171, 546, 812, 1362, 1381, 1385, 2816, 3038, 4034, 4307, 5015, 5117, 5118, 5124, 5176, 5196, 5541, 5687, 5708, 5709, 5710, 5875, 5876, 5939, 6085, 6516 and/or 7385.

The invention also provides nucleic acid encoding polypeptides of the invention. Such nucleic acids include those encoding the proteolytic products of the viral polyprotein (e.g. SEQ ID NOS: 3 to 13). Where such polypeptides do not include a native N-terminal methionine then it may be necessary to introduce an artificial one e.g. on its own or with a leader peptide.

Nucleic acids of the invention can be used in hybridisation reactions (e.g. Northern or Southern blots, or in nucleic acid microarrays or ‘gene chips’) and amplification reactions (e.g. PCR, SDA, SSSR, LCR, TMA, NASBA, etc.) and other nucleic acid techniques. They can also be used for polypeptide expression.

Nucleic acid according to the invention can take various forms (e.g. single-stranded, double-stranded, vectors, primers, probes, labeled, etc.). Nucleic acids of the invention may be circular or branched, but will generally be linear. Unless otherwise specified or required, any embodiment of the invention that utilizes a nucleic acid may utilize the double-stranded form and/or each of two complementary single-stranded forms which make up the double-stranded form. Primers and probes are generally single-stranded, as are antisense nucleic acids.

Nucleic acids of the invention are preferably provided in purified or substantially purified form i.e. substantially free from other nucleic acids (e.g. free from naturally-occurring nucleic acids), particularly from other viral or human nucleic acids, generally being at least about 50% pure (by weight), and usually at least about 90% pure. Nucleic acids of the invention are preferably coxsackie virus nucleic acids.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acid of the invention may be attached to a solid support (e.g. a bead, plate, filter, film, slide, microarray support, resin, etc.). Nucleic acid of the invention may be labelled e.g. with a radioactive or fluorescent label, or a biotin label. This is particularly useful where the nucleic acid is to be used in detection techniques e.g. where the nucleic acid is a primer or as a probe.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap. Where a nucleic acid of the invention has a uracil base at the 5′ terminus (e.g. SEQ ID NO: 1), the uracil may be covalently attached to a VPg protein.

Nucleic acids of the invention comprise sequences derived from SEQ ID NO: 1 or SEQ ID NO: 14, but they may also comprise additional sequences (e.g. in nucleic acids of formula 5′-X-Y-Z-3′, as defined above). This is particularly useful for primers, which may thus comprise a first sequence complementary to a coxsackie virus nucleic acid target and a second sequence which is not complementary to the nucleic acid target. Any such non-complementary sequences in the primer are preferably 5′ to the complementary sequences. Typical non-complementary sequences comprise restriction sites or promoter sequences.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T).

For certain embodiments of the invention, nucleic acids are preferably at least 7 nucleotides in length (e.g. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 90, 100, 110, 120, 130, 140, 150, 160, 170, 180, 190, 200, 225, 250, 275, 300 nucleotides or longer).

For certain embodiments of the invention, nucleic acids are preferably at most 500 nucleotides in length (e.g. 450, 400, 350, 300, 250, 200, 150, 140, 130, 120, 110, 100, 90, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15 nucleotides or shorter).

Primers and probes of the invention, and other nucleic acids used for hybridization, are preferably between 10 and 30 nucleotides in length (e.g. 10, 11, 12, 13, 14, 15, 16, 17, 18, 19, 20, 21, 22, 23, 24, 25, 26, 27, 28, 29, or 30 nucleotides). Such primers include SEQ ID NOS: 17 to 95.

References to a percentage sequence identity between two nucleic acid sequences mean that, when aligned, that percentage of bases are the same in comparing the two sequences. This alignment and the percent homology or sequence identity can be determined using software programs known in the art, for example those described in section 7.7.18 of reference 18. A preferred alignment program is GCG Gap (Genetics Computer Group, Wisconsin, Suite Version 10.1), preferably using default parameters, which are as follows: open gap=3; extend gap=1.

Where a nucleic acid is said to “encode” a polypeptide, this does not necessarily imply that it is translated, but it will include a series of codons which encode the amino acids of the polypeptide.

The invention provides a process for detecting nucleic acid of the invention, comprising the steps of: (a) contacting a nucleic probe according to the invention with a biological sample under hybridising conditions to form duplexes; and (b) detecting said duplexes.

The invention provides a process for detecting coxsackie virus in a biological sample (e.g. blood), comprising the step of contacting nucleic acid according to the invention with the biological sample under hybridising conditions. The process may involve nucleic acid amplification (e.g. PCR, SDA, SSSR, LCR, TMA, NASBA, etc.) or hybridisation (e.g. microarrays, blots, hybridisation with a probe in solution, etc.).

The invention also provides a virion comprising a RNA genome, wherein the RNA genome is a nucleic acid of the invention (e.g. comprising SEQ ID NO: 1).

### Polypeptides

The invention provides a polypeptide comprising an amino acid sequence that is a fragment of at least j contiguous amino acids of an amino acid sequence selected from SEQ ID NOS: 2 to 13. It also provides a polypeptide comprising an amino acid sequence (e.g. an amino acid sequence at least j amino acids long) that has at least b % sequence identity to an amino acid sequence selected from SEQ ID NOS: 2 to 13. It also provides a polypeptide comprising (i) an amino acid sequence that has at least b % sequence identity to an amino acid sequence selected from SEQ ID NOS: 2 to 13 and (ii) an amino acid sequence that is a fragment of at least j contiguous amino acids of an amino acid sequence selected from SEQ ID NOS: 2 to 13.

The percentage value of b is typically at least 50 e.g. 50, 55, 60, 65, 70, 75, 80, 85, 90, 91, 92, 93, 94, 95, 96, 97, 98, 99, 99.5, 99.9 or 100. Thus polypeptides of the invention include homologs, orthologs, allelic variants and functional mutants of SEQ ID NO: 2. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty=12 and gap extension penalty=1.

The value of j is typically at least 6 e.g. 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 60, 70, 80, 90, 100 or more.

The invention also provides a polypeptide of formula -XX-YY-ZZ-, wherein: -XX- is a sequence consisting of xx amino acids; -ZZ- is a sequence consisting of zz amino acids; -YY- is a sequence consisting of a fragment of an amino acid sequence selected from SEQ ID NOS: 2 to 13, provided that the amino acid sequence of -XX-YY-ZZ- is not a fragment of SEQ ID NO: 2. The value of xx+zz is at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100 etc.) i.e. either xx or zz may be zero. It is preferred that the value of xx+yy+zz is at least 8 (e.g. at least 9, 10, 11, 12, 13, 14, 15, 16, 17, 18, 19, 20, 21, 22, 23, 24, 25, 30, 35, 40, 45, 50, 60, 70, 80, 90, 100 etc.). It is preferred that the value of xx+yy+zz is at most 500 (e.g. at most 450, 400, 350, 300, 250, 200, 190, 180, 170, 160, 150, 140, 130, 120, 110, 100, 90, 80, 70, 60, 50, 40, 30, 25, 20, 19, 18, 17, 16, 15, 14, 13, 12, 11, 10, 9, 8).

Polypeptide of the invention may, compared to SEQ ID NO: 2, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cystine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may also include one or more (e.g. 1, 2, 3, 4, 5, 6, 7, 8, 9, 10, etc.) single amino acid deletions relative to SEQ ID NO: 2. The polypeptides may also include one or more (e.g. 1, 2, 3, 4, 5, 6, 7, 8, 9, 10, etc.) insertions (e.g. each of 1, 2, 3, 4 or 5 amino acids) relative to SEQ ID NO: 2.

A fragment of SEQ ID NO: 2 may comprise at least one T-cell or, preferably, a B-cell epitope of the sequence. T- and B-cell epitopes can be identified empirically (e.g. using PEPSCAN [19,20] or similar methods), or they can be predicted (e.g. using the Jameson-Wolf antigenic index [21], matrix-based approaches [22], TEPITOPE [23], neural networks [24], OptiMer & EpiMer [25, 26], ADEPT [27], Tsites [28], hydrophilicity [29], antigenic index [30] or the methods disclosed in reference 31 etc.).

Preferred fragments of polyprotein SEQ ID NO: 2 are SEQ ID NOS: 3, 4, 5, 6, 7, 8, 9, 10, 11, 12 and 13, which are its natural proteolytic derivatives. Other useful fragments include one or more of the following amino acids (numbered according to SEQ ID NO: 2): 207, 213, 214, 1458, 1459, 1461, 1478, 1485, 1656, 1711, 1781 and/or 1925. Polypeptides VP2, 3A, 3C and 3D are thus particularly useful for testing.

The invention provides mixtures of at least two polypeptides of the invention. For instance, it provides a mixture of 2, 3 or 4 of VP1, VP2, VP3 and/or VP4 (e.g. SEQ ID NOS: 3 to 6). These four proteins may be assembled as a protomer.

Polypeptides of the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides <40 amino acids long involves in vitro chemical synthesis [32,33]. Solid-phase peptide synthesis is particularly preferred, such as methods based on tBoc or Fmoc [34] chemistry. Enzymatic synthesis [35] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [36]. Where D-amino acids are included, however, it is preferred to use chemical synthesis. Polypeptides of the invention may have covalent modifications at the C-terminus and/or N-terminus.

Polypeptides of the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides of the invention are preferably provided in purified or substantially purified form i.e. substantially free from other polypeptides (e.g. free from naturally-occurring polypeptides), particularly from other coxsackie viral or human polypeptides, and are generally at least about 50% pure (by weight), and usually at least about 90% pure i.e. less than about 50%, and more preferably less than about 10% (e.g. 5%) of a composition is made up of other expressed polypeptides. Polypeptides of the invention are preferably coxsackie virus polypeptides.

Polypeptides of the invention may be attached to a solid support. Polypeptides of the invention may comprise a detectable label (e.g. a radioactive or fluorescent label, or a biotin label).

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains. Polypeptides of the invention can be naturally or non-naturally glycosylated (i.e. the polypeptide has a glycosylation pattern that differs from the glycosylation pattern found in the corresponding naturally occurring polypeptide).

Polypeptides of the invention are generally at least 7 amino acids in length (e.g. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 90, 100, 110, 120, 130, 140, 150, 160, 170, 180, 190, 200, 225, 250, 275, 300 amino acids or longer).

For certain embodiments of the invention, polypeptides are preferably at most 500 amino acids in length (e.g. 450, 400, 350, 300, 250, 200, 150, 140, 130, 120, 110, 100, 90, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15 amino acids or shorter).

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of reference 18. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is taught in reference 37.

### Antibodies

The invention provides antibody that binds to a polypeptide of the invention. Preferred antibodies of the invention recognize an epitope within SEQ ID NO: 2.

Antibodies of the invention may be polyclonal or monoclonal.

Antibodies of the invention may be produced by any suitable means e.g. by recombinant expression, or by administering (e.g. injecting) a polypeptide of the invention to an appropriate animal (e.g. a rabbit, hamster, mouse or other rodent).

Antibodies of the invention may include a label. The label may be detectable directly, such as a radioactive or fluorescent label. Alternatively, the label may be detectable indirectly, such as an enzyme whose products are detectable (e.g. luciferase, β-galactosidase, peroxidase, etc.).

Antibodies of the invention may be attached to a solid support.

In general, antibodies of the invention are provided in a non-naturally occurring environment e.g. they are separated from their naturally-occurring environment. In certain embodiments, the antibodies are present in a composition that is enriched for them as compared to a control. Antibodies of the invention are thus preferably provided in isolated or substantially isolated form i.e. the antibody is present in a composition that is substantially free of other antibodies, where by substantially free is meant that less than 75% (by weight), preferably less than 50%, and more preferably less than 10% (e.g. 5%) of the composition is made up of other antibodies.

The term “antibody” includes any suitable natural or artificial immunoglobulin or derivative thereof. In general, the antibody will comprise a Fv region which possesses specific antigen-binding activity. This includes, but is not limited to: whole immunoglobulins, antigen-binding immunoglobulin fragments (e.g. Fv, Fab, F(ab′)₂ etc.), single-chain antibodies (e.g. scFv), chimeric antibodies, humanized antibodies, veneered antibodies, etc.

To increase compatibility with the human immune system, the antibodies may be chimeric or humanized (e.g. refs. 38 & 39), or fully human antibodies may be used. Because humanized antibodies are far less immunogenic in humans than the original non-human monoclonal antibodies, they can be used for the treatment of humans with far less risk of anaphylaxis.

Humanized antibodies may be achieved by a variety of methods including, for example: (1) grafting non-human complementarity determining regions (CDRs) onto a human framework and constant region (“humanizing”), with the optional transfer of one or more framework residues from the non-human antibody; (2) transplanting entire non-human variable domains, but “cloaking” them with a human-like surface by replacement of surface residues (“veneering”). In the present invention, humanized antibodies will include both “humanized” and “veneered” antibodies. (refs. 40 to 46). CDRs are amino acid sequences which together define the binding affinity and specificity of a Fv region of a native immunoglobulin binding site [47,48]. Humanized or fully-human antibodies can also be produced using transgenic animals that are engineered to contain human immunoglobulin loci e.g. the “xeno-mouse” from Abgenix [49]. Phage display can also be used to select antibodies.

The phrase “constant region” refers to the portion of the antibody molecule that confers effector functions. In chimeric antibodies, mouse constant regions are substituted by human constant regions. The constant regions of humanized antibodies are derived from human immunoglobulins. The heavy chain constant region can be selected from any of the 5 isotypes: alpha, delta, epsilon, gamma or mu, and thus antibody can be of any isotype (e.g. IgG, IgA, IgM, IgD, IgE). IgG is preferred, which may be of any subclass (e.g. IgG₁, IgG₂).

### Nucleic Acid Amplification Methods

Nucleic acid in a sample can conveniently and sensitively be detected by nucleic acid amplification techniques such as PCR, SDA, SSSR, LCR, TMA, NASBA, T7 amplification, etc. The technique preferably gives exponential amplification. A preferred technique for use with RNA is RT-PCR (e.g. see chapter 15 of ref. 50). The technique may be quantitative and/or real-time.

Amplification techniques generally involve the use of two primers. Where a target sequence is single-stranded, the techniques generally involve a preliminary step in which a complementary strand is made in order to give a double-stranded target, thereby facilitating exponential amplification. The two primers hybridize to different strands of the double-stranded target and are then extended. The extended products can serve as targets for further rounds of hybridization/extension. The net effect is to amplify a template sequence within the target, the 5′ and 3′ termini of the template being defined by the locations of the two primers in the target.

The invention provides a kit comprising primers for amplifying a template sequence contained within a coxsackie virus nucleic acid target, the kit comprising a first primer and a second primer, wherein the first primer comprises a sequence substantially complementary to a portion of said template sequence and the second primer comprises a sequence substantially complementary to a portion of the complement of said template sequence, wherein the sequences within said primers which have substantial complementarity define the termini of the template sequence to be amplified.

The first primer and/or the second primer may include a detectable label (e.g. a fluorescent label, a radioactive label, etc.).

Primers may include a sequence that is not complementary to said template nucleic acid. Such sequences are preferably upstream of (i.e. 5′ to) the primer sequences, and may comprise a restriction site [51], a promoter sequence [52], etc.

A primer may terminate 0-10 nucleotides upstream of one of the following nucleotides, such that primer extension will incorporate the corresponding nucleotide (numbered according to SEQ ID NO: 1): 136, 137, 171, 546, 812, 1362, 1381, 1385, 2816, 3038, 4034, 4307, 5015, 5117, 5118, 5124, 5176, 5196, 5541, 5687, 5708, 5709, 5710, 5875, 5876, 5939, 6085, 6516, 7385.

Kits of the invention may further comprise a probe which is substantially complementary to the template sequence and/or to its complement and which can hybridize thereto. This probe can be used in a hybridization technique to detect amplified template.

Kits of the invention may further comprise primers and/or probes for generating and detecting an internal standard, in order to aid quantitative measurements [53].

Kits of the invention may comprise more than one pair of primers (e.g. for nested amplification), and one primer may be common to more than one primer pair. The kit may also comprise more than one probe.

The template sequence is preferably at least 50 nucleotides long (e.g. 60, 70, 80, 90, 100, 125, 150, 175, 200, 250, 300, 400, 500, 600, 700, 800, 900, 1000, 1250, 1500, 2000, 3000 nucleotides or longer). The length of the template is inherently limited by the length of the target within which it is located, but the template sequence is preferably shorter than 500 nucleotides (e.g. 450, 400, 350, 300, 250, 200, 175, 150, 125, 100, 90, 80, 70, or shorter).

The template sequence may be any part of a coxsackie virus genome sequence.

Specific primers that have been used for CBV4 amplification are SEQ ID NOS: 17 to 95, where SEQ ID NOS: 17 to 56 are forward primers and 57 to 95 are reverse primers.

The invention provides a process for preparing a fragment of a target sequence, wherein the fragment is prepared by extension of a nucleic acid primer. The target sequence and/or the primer are nucleic acids of the invention. The primer extension reaction may involve nucleic acid amplification (e.g. PCR, SDA, SSSR, LCR, TMA, NASBA, etc.).

### Pharmaceutical Compositions

The invention provides a pharmaceutical composition comprising an antiviral, nucleic acid, polypeptide, or antibody of the invention. The invention also provides their use as medicaments (e.g. for prevention and/or treatment of type 1 diabetes), and use of the components in the manufacture of medicaments for treating prostate cancer. The invention also provides a method for raising an immune response, comprising administering an immunogenic dose of nucleic acid or polypeptide of the invention to an animal (e.g. to a patient).

Pharmaceutical compositions encompassed by the present invention include as active agent, an antiviral, nucleic acid, polypeptide, and/or antibody of the invention disclosed herein in a therapeutically effective amount. An “effective amount” is an amount sufficient to effect beneficial or desired results, including clinical results. An effective amount can be administered in one or more administrations. For purposes of this invention, an effective amount is an amount that is sufficient to palliate, ameliorate, stabilize, reverse, slow or delay the symptoms and/or progression of type 1 diabetes.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers (e.g. insulin production). Therapeutic effects also include reduction in physical symptoms. The precise effective amount for a subject will depend upon the subject's size and health, the nature and extent of the condition, and the therapeutics or combination of therapeutics selected for administration. The effective amount for a given situation is determined by routine experimentation and is within the judgment of the clinician. For purposes of the present invention, an effective dose will generally be from about 0.01 mg/kg to about 5 mg/kg, or about 0.01 mg/ kg to about 50 mg/kg or about 0.05 mg/kg to about 10 mg/kg of the compositions of the present invention in the individual to which it is administered.

A pharmaceutical composition can also contain a pharmaceutically acceptable carrier. A thorough discussion of such carriers is available in reference 54.

Once formulated, the compositions contemplated by the invention can be (1) administered directly to the subject (e.g. as nucleic acid, polypeptides, small molecule antivirals, and the like); or (2) delivered ex vivo, to cells derived from the subject (e.g. as in ex vivo gene therapy). Direct delivery of the compositions will generally be accomplished by parenteral injection, e.g. subcutaneously, intraperitoneally, intravenously or intramuscularly, intratumoral or to the interstitial space of a tissue. Other modes of administration include oral and pulmonary administration, suppositories, and transdermal applications, needles, and gene guns or hyposprays. Dosage treatment can be a single dose schedule or a multiple dose schedule.

### General

The term “comprising” encompasses “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may thus be omitted from the definition of the invention.

### BRIEF DESCRIPTION OF DRAWINGS

FIGS. 1 and 2 show polyprotein processing for coxsackie virus (adapted from ref. 15). The SEQ ID NOs of proteolytic fragments for the Tuscany strain of CBV4 are shown.

FIG. 3 is a dendrogram of polyprotein sequences from coxsackievirus viruses A9-Griggs, B3-Nancy, B4-E2, B4-JVB and B5-Faulkner.

### MODES FOR CARRYING OUT THE INVENTION

Certain aspects of the present invention are described in greater detail in the non-limiting examples that follow. The examples are put forth so as to provide those of ordinary skill in the art with a disclosure and description of how to make and use the present invention, and are not intended to limit the scope of what the inventors regard as their invention nor are they intended to represent that the experiments below are all and only experiments performed. Efforts have been made to ensure accuracy with respect to numbers used (e.g. amounts, temperature, etc.) but some experimental errors and deviations should be accounted for.

Whole pancreases were obtained from donors with recent onset type 1 diabetes, one 26-yr-old recipient of a whole pancreas graft and from 26 normal caucasoid multiorgan donors with no family history of type 1 or type 2 diabetes. One of the diabetic patients was a caucasoid type 1 diabetic woman recipient of a whole pancreas graft which, at the time of removal, showed only partial islet function. Six patients (#1 to #6) were studied in detail.

Pancreatic specimens were frozen in liquid nitrogen or formalin-fixed and paraffin-embedded for immunohistochemical investigations.

Investigation revealed that patients were suffering from non-destructive insulitis with NK cells infiltration. In patients #1-3, the mononuclear cell infiltrate was composed mainly of CD94-positive (NK) cells and, to a lesser extent, of T lymphocytes, with occasional B-lymphocytes and CD68+ cells. In contrast, in cases #4-6 NK cells were not observed amongst the moderate infiltrates of CD45RO+ cells. No double-positive cells for CD94 and CD45RO were detected in any of the pancreas analyzed, thus confirming that CD94-positive cells, where observed, were indeed NK cells and did not belong to the small subset of T-lymphocytes that may express CD94. IFNα-positive cells were detected in pancreatic islets from patients #1-3 but not from patients #4-6 or from any control pancreata, suggesting ongoing or previous islet viral infection.

Islets were prepared by intraductal collagenase solution injection and density gradient purification, and β-cells were shown to be specifically infected by enteroviruses. Expression of capsid protein VP1 was checked, and strong staining was observed in the majority of pancreatic islets of patients #1-3 and also in a few scattered exocrine cells. This VP1 positive immunostaining was associated with a NK-dominated mild insulitis. No VP1 was detected in pancreatic sections from control organ donors. VP1 colocalized with insulin, but not glucagon, indicating a β-cell specific enterovirus tropism in the pancreatic islets. Most insulin-positive cells stained positive for VP1.

Using electron microscopy, viral inclusions were specifically located in the cytoplasm of pancreatic β-cells of VP1-positive islets, without alterations in α or δ cells. The percentage of infected β-cells, determined by electron microscopy, ranged from 76% to 88%. Varying degrees of cytopathic effects were observed, from almost intact cells to organelle disruption and cellular membrane damage, although no morphological sign of apoptosis was seen. Virus particles were abundant in areas close to mitochondria, many of which appeared swollen or severely damaged. Approximately 40% of β-cells showed distorted and wrinkled nuclei, suggestive of pyknosis. Conversely, no viral inclusions were detected in pancreatic sections from any of the control organ donors.

By studying insulin secretion in response to glucose and other secretagogues, infected islets were shown in vitro to have lost β-cell function. Islets isolated from two infected pancreata (patients #1 and #2) and from 3 age-matched healthy control glands were analyzed and, while insulin content was similar in diabetic and control islets, insulin release in response to glucose, arginine and glibenclamide was significantly lower (96-98% lower) from islets obtained from diabetic glands compared to control islets.

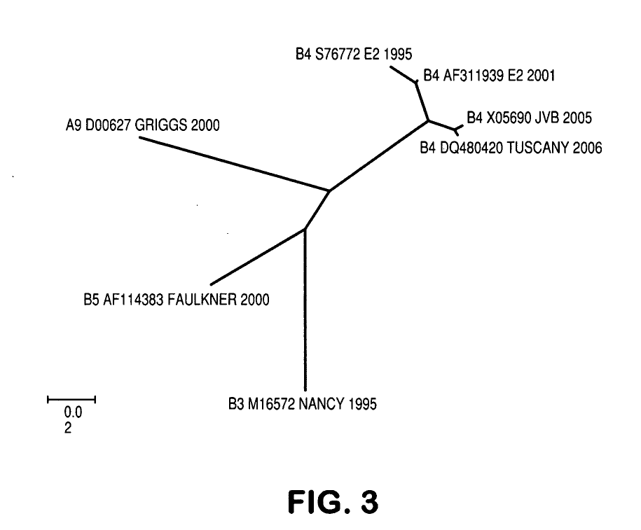

Virus was extracted from islets of patient #2 and subjected to whole genome sequencing. An unambiguous viral genome sequence of 7395 nt was assembled (SEQ ID NO: 1). This genome encodes polyprotein SEQ ID NO: 2. This polyprotein sequence was analysed against homologues from coxsackie virus strains previously shown to be able to in vitro infect human islets (Coxsackie A9-Griggs, B3-Nancy, B4-E2, B4-JVB, B5-Faulkner), and sequences were aligned to build a maximum likelihood phylogeny. The tree topology and branch lengths are highly conserved (FIG. 3), with the closest match being B4-JVB (genome SEQ ID NO: 15, encoding SEQ ID NO: 16).

An alignment of the DNA forms of SEQ ID NOs: 1 and 15 is shown below:

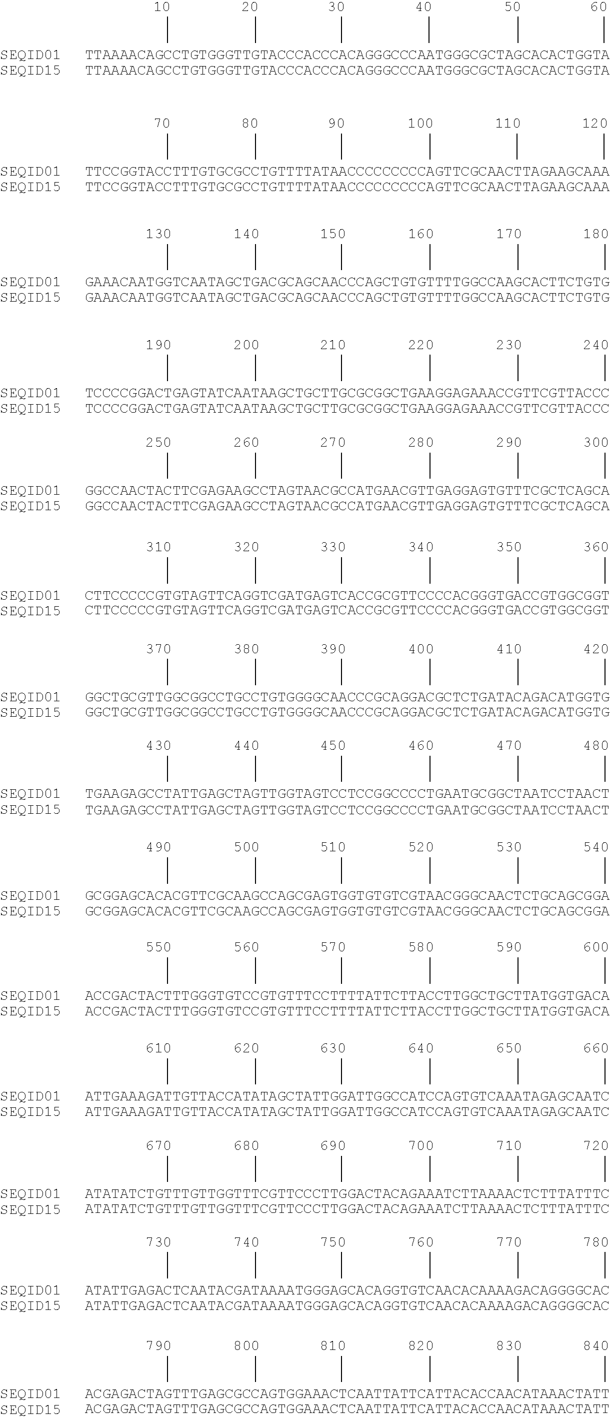

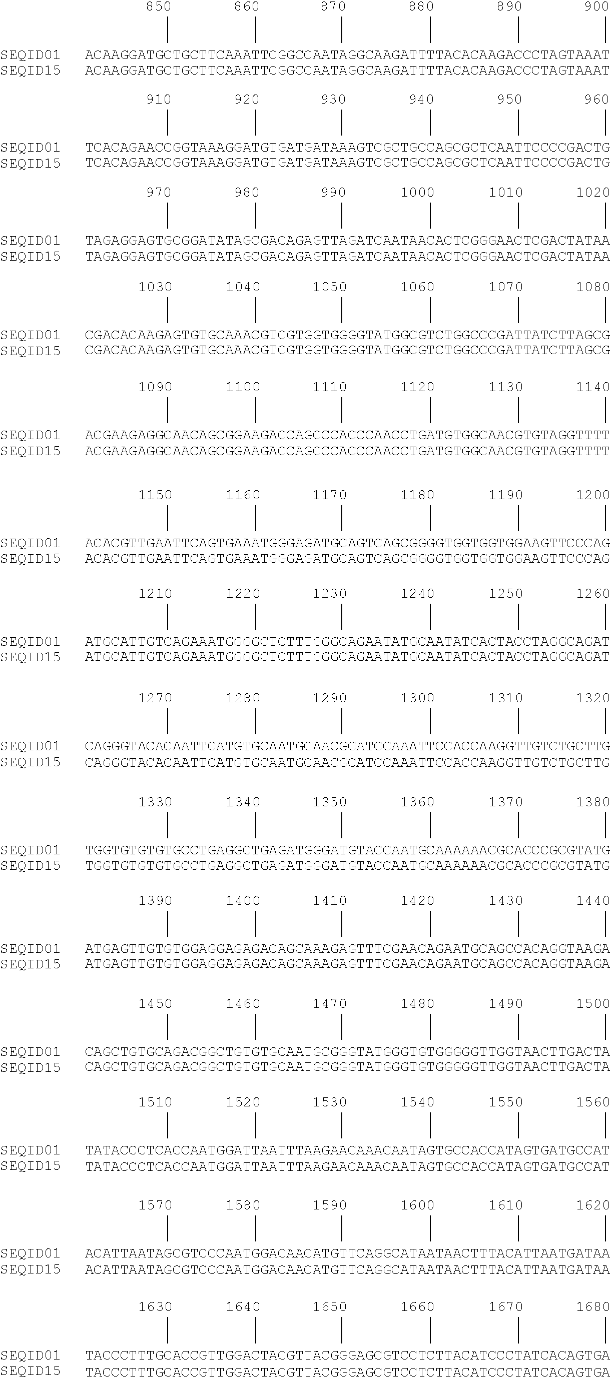

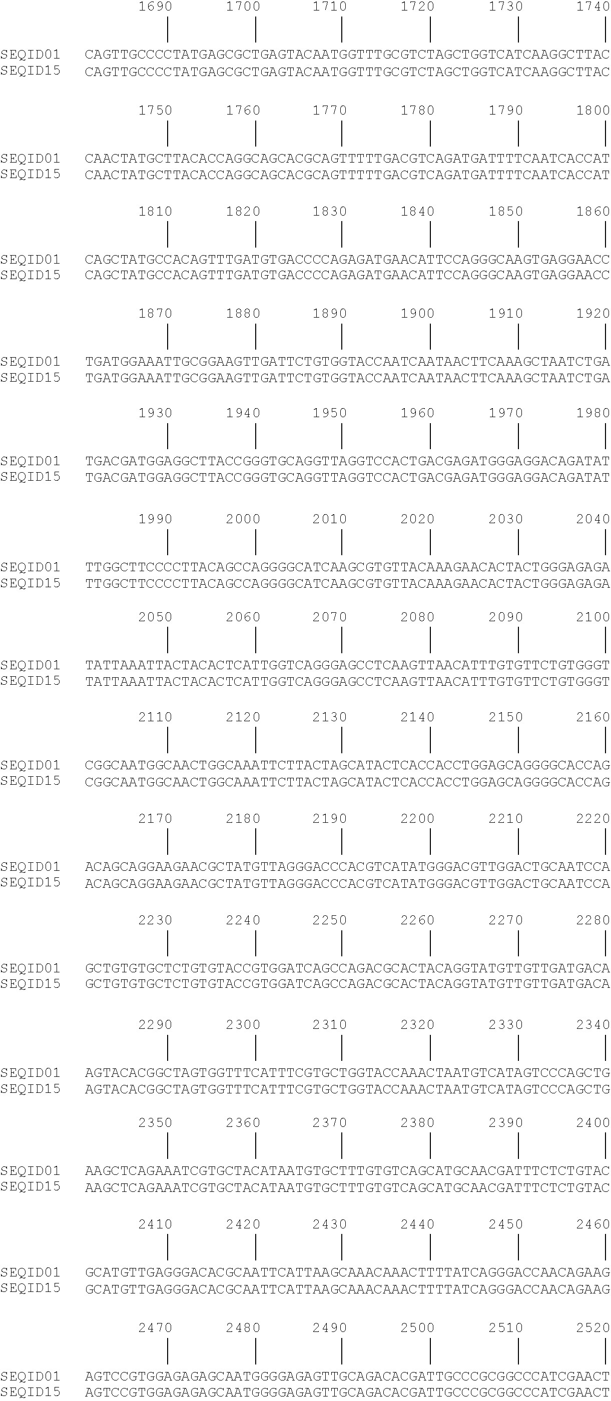

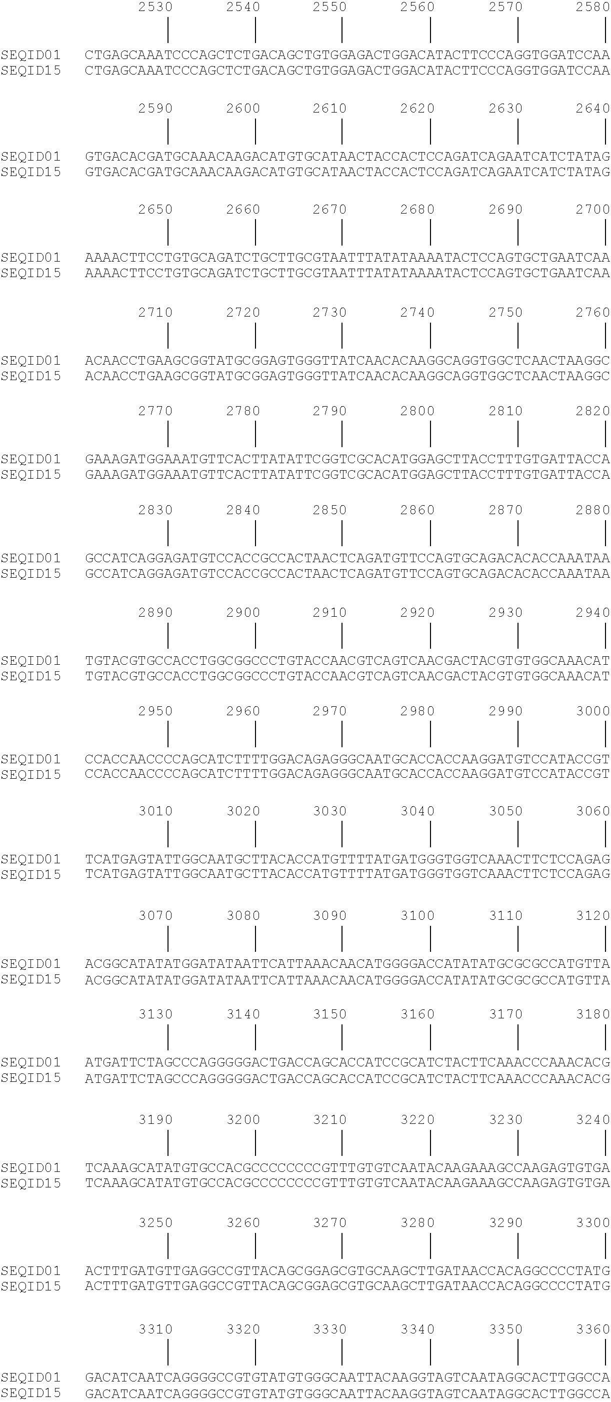

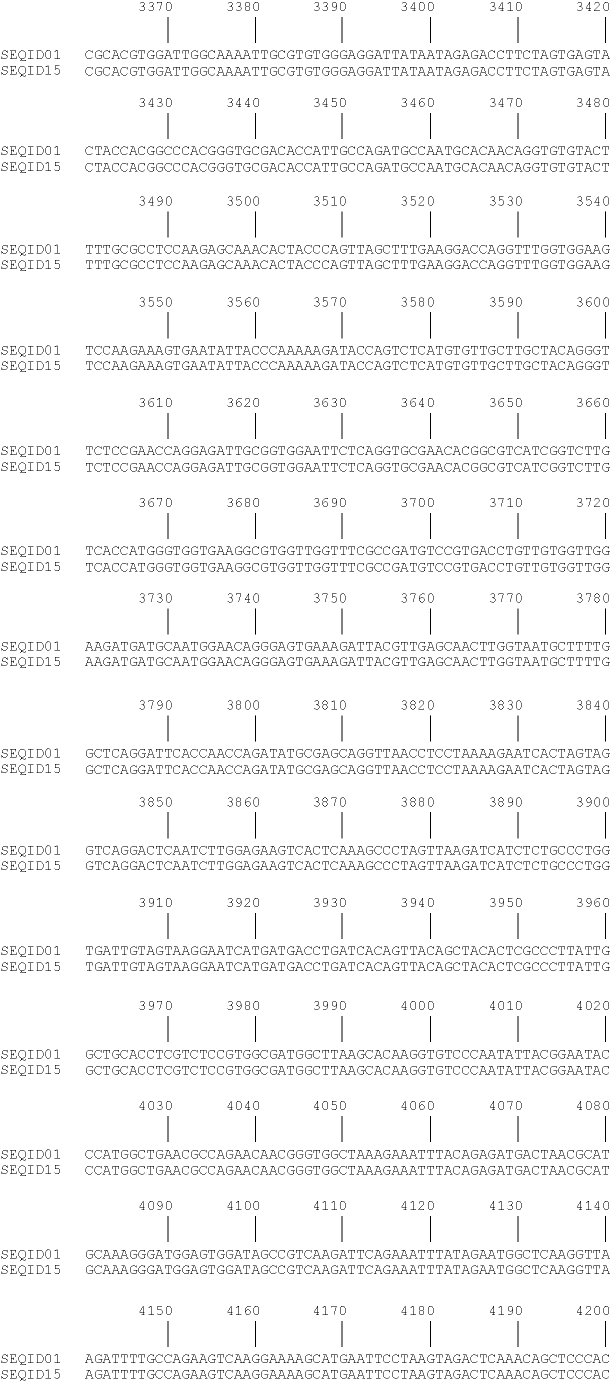

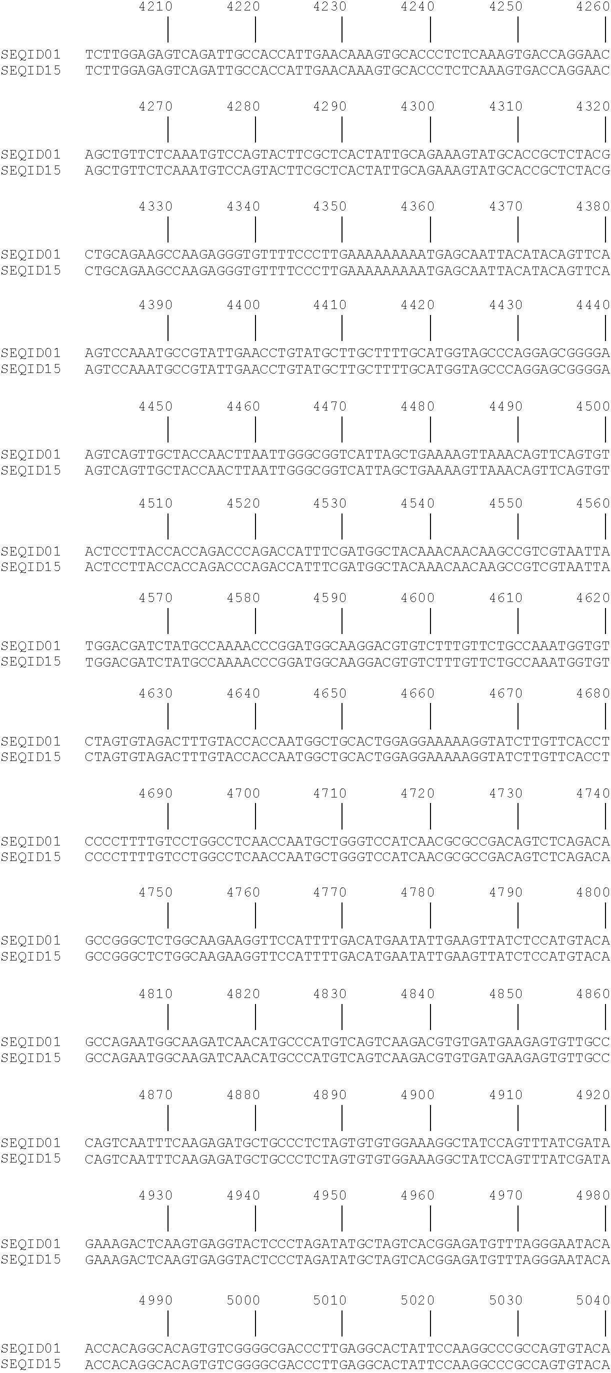

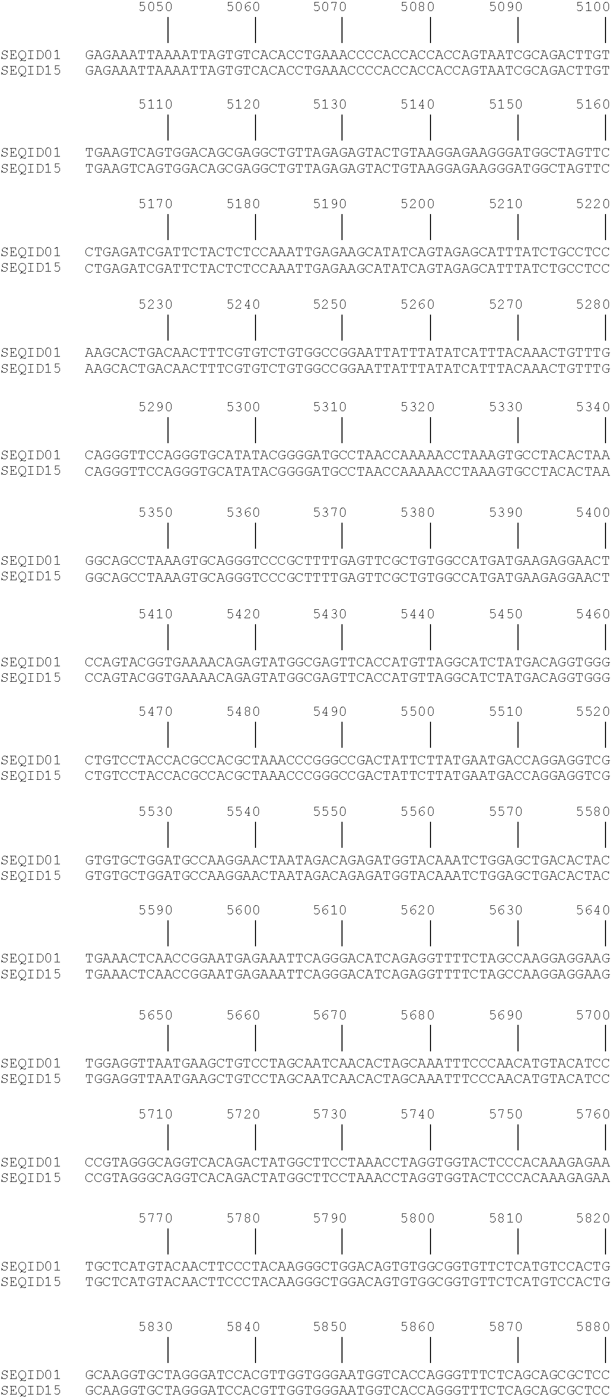

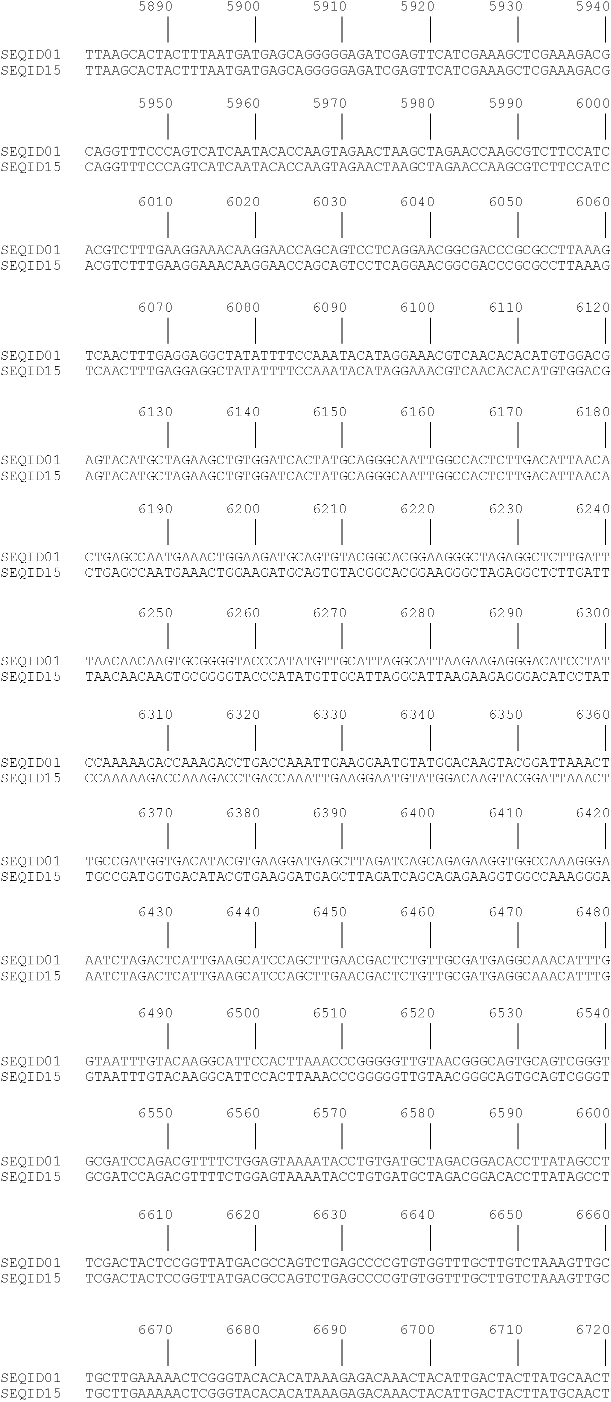

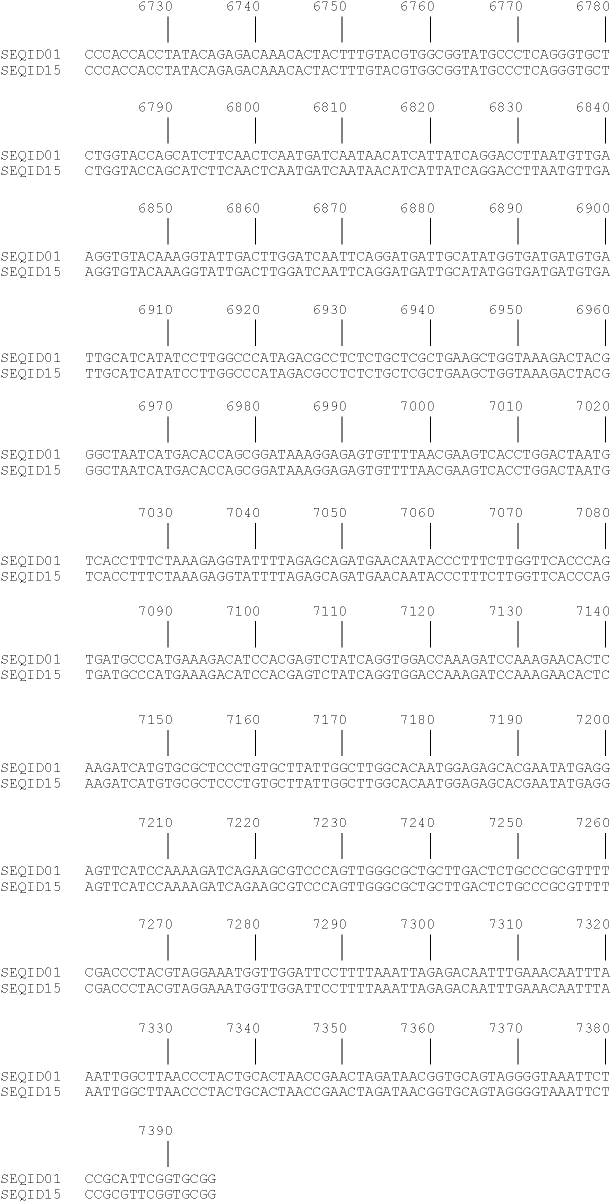

Both genomes are 7395mers. There are 29 differences, representing an overall sequence identity of 99.61%. The differences are as follows:

| | 136 | 137 | 171 | 546 | 812 | 1362 | 1381 | 1385 | 2816 | 3038 |
|---|---|---|---|---|---|---|---|---|---|---|
| Region | VP4 | VP4 | VP4 | VP2 | VP2 | VP3 | VP3 | VP3 | 2A | 2B |
| SEQ1 | A | G | C | C | A | A | A | G | C | T |
| SEQ15 | T | A | T | G | G | G | G | T | T | C |

| | 4034 | 4307 | 5015 | 5117 | 5118 | 5124 | 5176 | 5196 | 5541 | 5687 |
|---|---|---|---|---|---|---|---|---|---|---|
| Region | 2C | 3A | 3C | 3C | 3C | 3C | 3D | 3D | 3D | 3D |
| SEQ1 | C | T | A | C | G | G | C | A | C | C |
| SEQ15 | G | C | G | G | C | A | T | G | T | A |

| | 5708 | 5709 | 5710 | 5875 | 5876 | 5939 | 6085 | 6516 | 7385 |
|---|---|---|---|---|---|---|---|---|---|
| Region | 3D | 3D | 3D | 3D | 3D | 3D | 3D | 3D | 3′UTR |
| SEQ1 | G | C | A | C | G | C | C | G | A |
| SEQ15 | C | A | G | G | C | T | T | A | G |

When translated, there are 12 differences between the two 2183-mer polyproteins, representing an overall sequence identity of 99.45%:

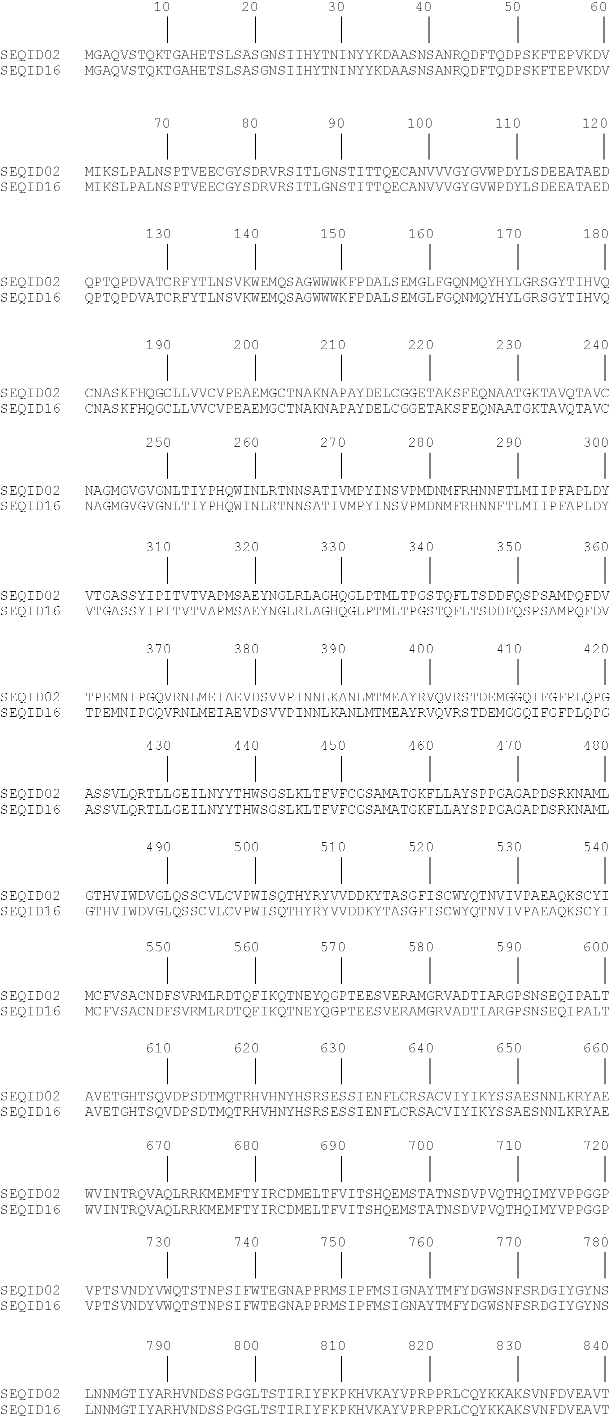

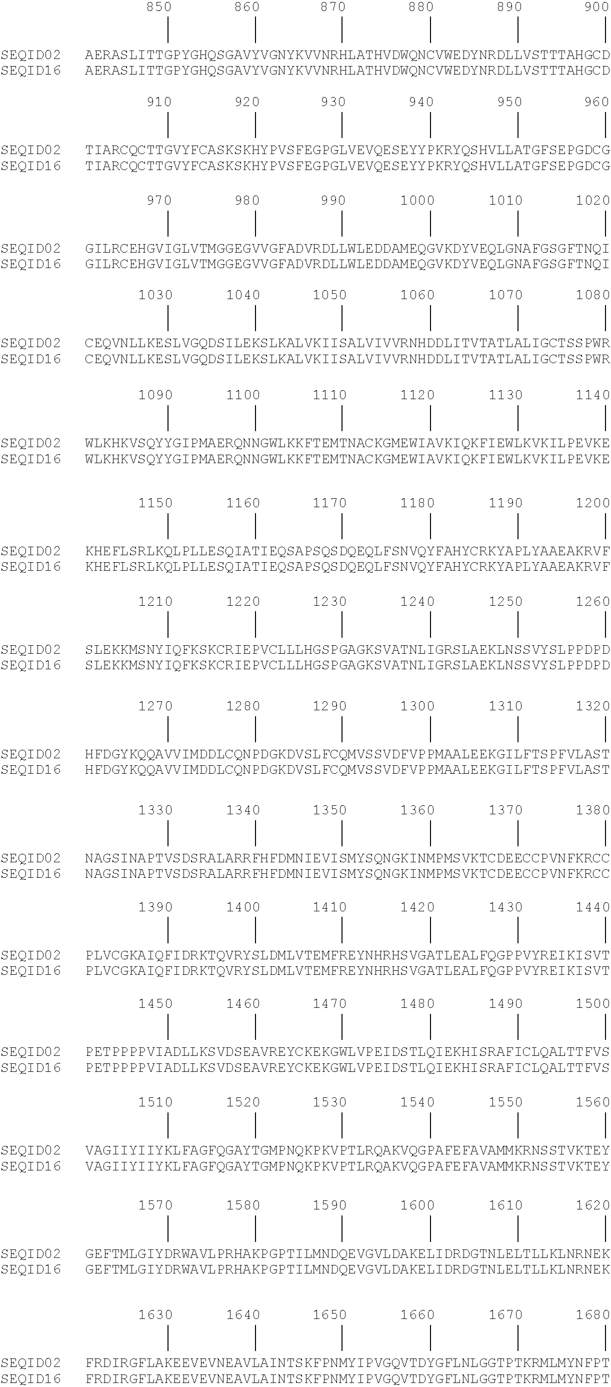

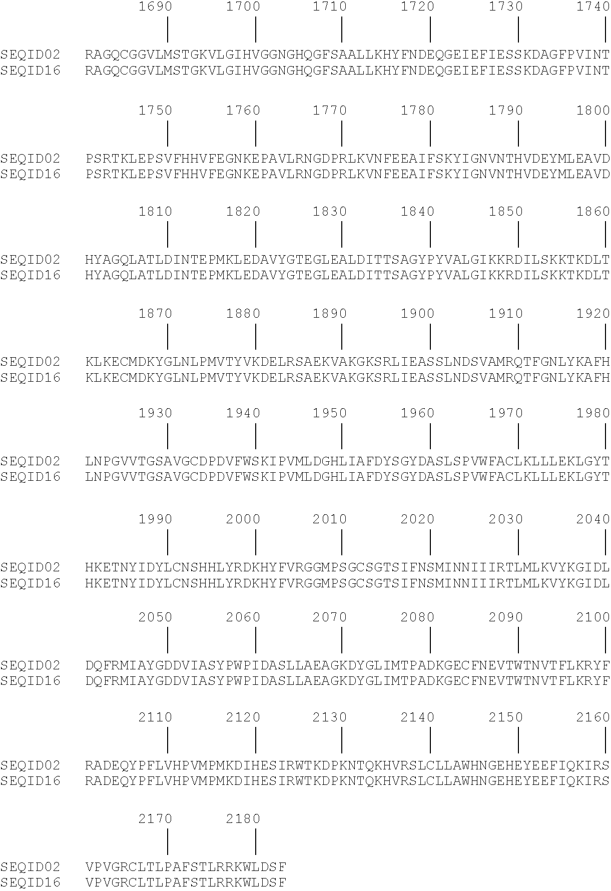

The differences are as follows:

| | 207 | 213 | 214 | 1458 | 1459 | 1461 |
|---|---|---|---|---|---|---|
| Region | VP2 | VP2 | VP2 | 3A | 3A | 3A |
| SEQ1 | K | D | E | S | E | V |
| SEQ15 | E | G | D | R | Q | I |

| | 1478 | 1485 | 1656 | 1711 | 1781 | 1925 |
|---|---|---|---|---|---|---|
| Region | 3A | 3A | 3C | 3C | 3D | 3D |
| SEQ1 | T | I | Q | A | S | V |
| SEQ15 | I | V | R | G | F | I |

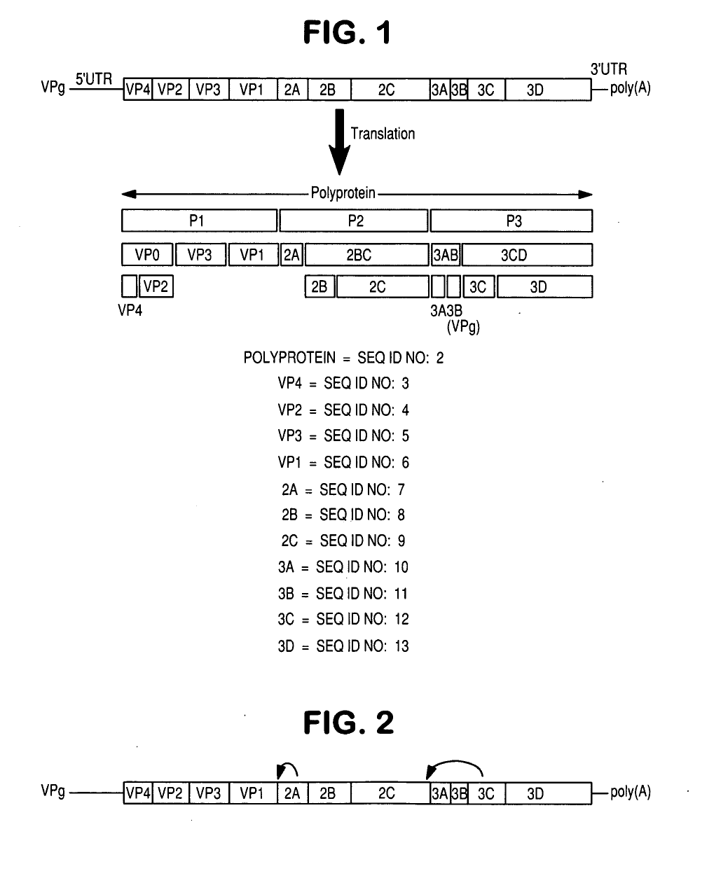

The polyprotein (SEQ ID NO: 2) is processed as shown in FIGS. 1 and 2 to give final proteins with amino acid sequences SEQ ID NOS: 3 to 13.

Virus was isolated from islets of patient #2 by homogenization and passaging in oral epidermoid carcinoma cells (KB cells). Virus was harvested from the initial KB infection, frozen in aliquots, and thawed and amplified on KB cells only one more time before being used to infect fresh human islets. Human islets prepared from 10 independent pancreata from non-diabetic organ donors were each separately co-cultured with virus-containing solution in M199 culture medium at 36° C. and checked after 4 and 7 days.

The isolated virus could infect human pancreatic beta-cells in vitro and impair their glucose-stimulated insulin secretion. By electron microscopy, viral inclusions were observed in the cytoplasm of 17±7% and 33±14% of beta-cells after 4 and 7 days of co-culture respectively. This finding was confirmed by VP-1 staining. Insulin secretion in response to glucose was assessed by static incubation method and perfusion procedure. Insulin secretion was severely impaired.

Cyotkine expression studies showed that infected and infiltrated islets express and secrete IL-10 and TNF-α. Islets isolated from 2 infected samples and from 3 age-matched healthy control glands were analyzed for cytokines. IL-4, IL-10, IFN-γ, TGF-β and TNF-α mRNA expression were analyzed by real time quantitative PCR, while cytokine secretion was determined by ELISA in cultured islets. IL-10 and TNF-α were the only cytokines detected in diabetic islets by both RT-PCR and ELISA. Q-PCR showed mRNA for IL-10 and TNF-α. IL-10 and TNF-α, but not IFN-γ, IL-4 or TGF-β, were detected by ELISA in supernatants of cultured diabetic islets. None of these cytokines were detectable in islets from the three non-diabetic control organ donors by RT-PCR, Q-PCR or by ELISA.

Cellular autoimmune responses were studied, but no T-cell autoreactivity was seen in patients' intra-islet lymphocytes. Responses were checked both in peripheral blood and intra-islet lymphocytes of patient #1. Autoreactivity in PBMC was restricted to the autoantigen IA-2, which mirrored the exclusive presence of autoantibodies against this β-cell determinant. A T-cell line was generated that was restricted by the disease predisposing HLA-DRB1*0401. The epitopes recognized included peptides previously identified as immunodominant epitopes and naturally processed peptides of IA-2. The cytokine production profile of these IA-2 specific autoreactive T-cells after primary stimulation was limited to TNF-α and substantial levels of IL-10. This anti-inflammatory cytokine profile matched the in situ cytokine expression in the insulitic islets and was accompanied by extremely high levels of circulating CD4+ T-cells with potentially regulatory phenotype.

Since both the recipient and the pancreas allograft expressed HLA-A2(0201), PBMCs were further tested for the presence of cytotoxic T-cells reactive with the autoantigenic peptide of insulin B-chain or a control peptide from human cytomegalovirus p65. 0.03% of CD3+CD8+ T-cells stained for the insulin-HLA-tetramer versus 0.64% of hCMV-HLA-tetramer binding cytotoxic T-cells, suggesting that the degree of cytotoxic T-cell autoreactivity was limited.

Islet-infiltrating leukocytes were cultured and expanded from islets isolated from the explanted pancreas allograft and tested for specificity for islet autoantigens or virus proteins. Despite good viability and strong reactivity to T-cell growth factor, none of these antigens were recognized, which is in accordance with the large percentage of NK cells in the infiltrates.

These results provide the first evidence of a relation between β-cell specific enterovirus infection, insulitis and β-cell dysfunction in human type 1 diabetes, with the identification of a Coxsackie-B4 virus which can persistently infect β-cells, interfering with function but without triggering cell destruction. The viral infection of β-cells together with the insulitic process could explain the impairment of insulin secretory function, and confirms previous in vitro studies on rat and human islet cells infected with different strains of Coxsackievirus, or exposed to IL-10 or to TNF-α [55-57]. In addition, the expression of only these two cytokines by the diabetic islets studied is in line with the lack of β-cell destruction, in the light of the findings on the protective effects of IL-10 on human islets in vitro [58] and of TNF-α on mouse islets in vivo [59], while IFN-γ was shown to be essential for destruction of β-cells in mice [60]. Furthermore, the insulitis does not seem to be directly pathogenic to β-cells, in spite of viral infection, as β-cell insulin content and proportion of β-cells per islet were similar in infected and in control islets, and no evidence of increased apoptosis was found. The absence of autoreactive T-cells amongst the infiltrating leukocytes, combined with an anti-inflammatory cytokine profile, could explain the lack of β-cell destruction. This would be in full accordance with findings in experimental autoimmune diabetes in mice, where enterovirus was shown to be diabetogenic only in case of a pre-existent autoimmune insulitis [61], while the response by β-cells could fundamentally determine their survival [62]. Coxsackie B3 infection has been shown to suppress proinflammatory cytokines and induce IL-10 production in host cells (e.g. human monocytes) as a potential strategy to perturb the anti-viral host activity leading to defective viral clearance and persistent infection [63].

In conclusion, the results herein demonstrate a correlation between β-cell selective enterovirus infection and a certain pattern of insulitis. This insulitis is dominated by NK cells, lacks islet autoreactivity, is non-destructive to β-cells and nevertheless causes β-cell dysfunction. Therefore, these findings imply that insulitis and autoimmunity are separate features and are both necessary for β-cell destruction, while insulitis in the absence of autoimmunity is not β-cell destructive. For cases showing viral infection of β-cells and a limited degree of islet autoreactivity there was an HLA phenotype that was distinct from HLA haplotypes associated with predisposition to type 1 diabetes. Together, these findings support the hypothesis that β-cell destruction requires autoimmunity with proinflammatory cytokine production, whereas viral infection by itself is not necessarily sufficient to cause this destruction. However, in a subset of type 1 diabetes patients, viral infection by itself does apparently lead to NK dominated insulitis, to β-cell dysfunction, and to a deficiency in insulin secretion with consequent hyperglycemia.

The above description of preferred embodiments of the invention has been presented by way of illustration and example for purposes of clarity and understanding. It is not intended to be exhaustive or to limit the invention to the precise forms disclosed. It will be readily apparent to those of ordinary skill in the art in light of the teachings of this invention that many changes and modifications may be made thereto without departing from the spirit of the invention. It is intended that the scope of the invention be defined by the appended claims and their equivalents.

### REFERENCES

### The Contents of which are Hereby Incorporated in Full by Reference

[1] Hyöty & Talyor (2002) Diabetologia 45:1353-61.
[2] Jun & Yoon (2001)Diabetologia 44(3):271-85.
[3] Green et al. (2004)Diabet Med 21(6):507-14.
[4] Berg et al. (2006)Antiviral Res “Antiviral treatment of Coxsackie B virus infection in human pancreatic islets”
[5] Gazidova et al. (2007) Bioorg Med. Chem. 2007 Jan. 15; 15(2):749-58.
[6] Tait et al. (2006) Antiviral Res. 72(3):252-5.
[7] Nikolaeva-Glomb & Galabov (2004) Antiviral Res 62(1):9-19
[8] Qi et al. (2000) Zhonghua Shi Yan He Lin Chuang Bing Du Xue Za Zhi. 14(3):253-6.
[9] Ahn et al. (2005) J. Virol. 79(13):8620-4.
[10] Fohlman et al. (1993) Scand J Infect Dis Suppl. 88:103-8.
[11] CN-1535725.
[12] CN-1772304
[13] CN-1772305.
[14] CN-1772306.
[15] Fields' Virology (4th edition) 2001, ISBN 0-7817-1832-5.
[16] Bolanaki et al. (2005) Mol Cell Probes 19(2):127-35.
[17] Mulders et al. (2000) J Gen Virol 81:803-12.
[18] Current Protocols in Molecular Biology (F. M. Ausubel et al. eds., 1987) Supplement 30.
[19] Geysen et al. (1984) PNAS USA 81:3998-4002.
[20] Carter (1994) Methods Mol Biol 36:207-23.
[21] Jameson, B A et al. 1988 , CABIOS 4(1):181-186.
[22] Raddrizzani & Hammer (2000) Brief Bioinform 1(2):179-89.
[23] De Lalla et al. (1999) J. Immunol. 163:1725-29.
[24] Brusic et al. (1998) Bioinformatics 14(2):121-30
[25] Meister et al. (1995) Vaccine 13(6):581-91.
[26] Roberts et al. (1996) AIDS Res Hum Retroviruses 12(7):593-610.
[27] Maksyutov & Zagrebelnaya (1993) Comput. Appl Biosci 9(3):291-7.
[28] Feller & de la Cruz (1991) Nature 349(6311):720-1.
[29] Hopp (1993) Peptide Research 6:183-190.
[30] Welling et al. (1985) FEBS Lett. 188:215-218.
[31] Davenport et al. (1995) Immunogenetics 42:392-297.
[32] Bodanszky (1993) Principles of Peptide Synthesis (ISBN: 0387564314).
[33] Fields et al. (1997) Meth Enzymol 289 : Solid-Phase Peptide Synthesis. ISBN: 0121821900.
[34] Chan & White (2000) Fmoc Solid Phase Peptide Synthesis. ISBN: 0199637245.
[35] Kullmann (1987) Enzymatic Peptide Synthesis. ISBN: 0849368413.
[36] Ibba (1996) Biotechnol Genet Eng Rev 13:197-216.
[37] Smith & Waterman (1981) Adv. Appl. Math. 2: 482-489.
[38] Breedveld (2000) Lancet 355(9205):735-740.
[39] Gorman & Clark (1990) Semin. Immunol. 2:457-466
[40] Jones et al. (1986) Nature 321:522-525.
[41] Morrison et al. (1984) Proc. Natl. Acad. Sci, U.S.A., 81:6851-6855.
[42] Morrison & Oi (1988) Adv. Immunol., 44:65-92.
[43] Verhoeyer et al. (1988) Science 239:1534-1536.
[44] Padlan (1991) Molec. Immun. 28:489-498.
[45] Padlan (1994) Molec. Immunol. 31(3):169-217.
[46] Kettleborough et al. (1991) Protein Eng. 4(7):773-83
[47] Chothia et al. (1987) J. Mol. Biol. 196:901-917.
[48] Kabat et al. U.S. Dept. of Health and Human Services NIH Publication No. 91-3242 (1991)
[49] Green (1999) J Immunol Methods. 231(1-2):11-23.
[50] Farrell (1998) RNA Methodologies (Academic Press; ISBN 0-12-249695-7).
[51] EP-B-0509612.
[52] EP-B-0505012.
[53] Fille et al. (1997) Biotechniques 23:34-36.
[54] Gennaro (2000) Remington: The Science and Practice of Pharmacy. 20th edition, ISBN: 0683306472.
[55] Roivainen et al. (2002) M. Diabetologia 45:693-702.
[56] Laffranchi & Spinas (1996) Eur. J Endocrinol. 135:374-378.
[57] Zhang & Kim (1995) FEBS Lett. 377:237-239.
[58] Marselli et al. (2001) J. Clin. Endocrinol. Metab 86:4974-4978.
[59] Christen et al. (2001) J. Immunol. 166:7023-7032.
[60] von Herrath & Oldstone (1997) J. Exp. Med. 185:531-539.
[61] Serreze et al. (2000) Diabetes 49:708-711.
[62] Flodstrom et al. (2002) Nat. Immunol. 3:373-382.
[63] Hofmann et al. (2001) J. Med. Virol. 64:487-498.

1. A method for preventing or treating type 1 diabetes in a patient, comprising a step of administering to the patient an antiviral compound effective against a coxsackie virus.

2. A method for preventing or treating type 1 diabetes in a patient, comprising a step of administering to the patient a composition that comprises a coxsackie virus immunogen.

3. A method for preventing or treating type 1 diabetes in a patient, comprising an immunomodulatory compound effective to inhibit natural killer cell activity.

4. An assay method comprising a step of detecting in a patient sample the presence or absence of (i) a coxsackie virus or an expression product thereof, and/or (ii) an immune response against a coxsackie virus.

5. The method of any preceding claim, wherein the virus is a group B coxsackie virus

6. The method of any preceding claim, wherein the virus is a type 4 group B coxsackie virus.

7. The method of any preceding claim, wherein the virus is the Tuscany B4 strain.

8. Nucleic acid comprising a nucleotide sequence that is a fragment of at least 6 contiguous nucleotides of SEQ ID NO: 1 or SEQ ID NO: 14.

9. Nucleic acid comprising a nucleotide sequence that has at least 80% sequence identity to SEQ ID NO: 1 or SEQ ID NO:

10. Nucleic acid of formula 5′-X-Y-Z-3′, wherein: -X- is a nucleotide sequence consisting of at least 1 nucleotide; -Z- is a nucleotide sequence consisting of at least 1 nucleotide; -Y- is a nucleotide sequence consisting of either (a) a fragment of SEQ ID NO: 1 or SEQ ID NO: 14, or (b) the complement of (a); and said nucleic acid 5′ X-Y-Z-3′ is neither (i) a fragment of SEQ ID NO: 1 or SEQ ID NO: 14 nor (ii) the complement of (i).

11. A polypeptide comprising an amino acid sequence that is a fragment of at least 6 contiguous amino acids of an amino acid sequence selected from SEQ ID NOS: 2 to 13.

12. A polypeptide comprising an amino acid sequence that has at least 80% sequence identity to an amino acid sequence selected from SEQ ID NOS: 2 to 13.

13. A polypeptide of formula -XX-YY-ZZ-, wherein: -XX- is a sequence consisting of at least 1 amino acid; -ZZ- is a sequence consisting of at least 1 amino acid; -YY- is a sequence consisting of a fragment of an amino acid sequence selected from SEQ ID NOS: 2 to 13, provided that the amino acid sequence of-XX-YY-ZZ- is not a fragment of SEQ ID NO: 2.

14. Antibody that binds to a polypeptide of claim 14 or claim 15.


SEQUENCE LISTING (95 sequences)

SEQ ID NO: 1 - RNA, 7395 nt, Coxsackie B4 virus
uuaaaacagccuguggguuguacccacccacagggcccaaugggcgcuagcacacugguauuccgguaccuuugugcgccuguuuuauaacccccccccaguucgcaacuuagaagcaaagaaacaauggucaauagcugacgcagcaacccagcuguguuuuggccaagcacuucuguguccccggacugaguaucaauaagcugcuugcgcggcugaaggagaaaccguucguuacccggccaacuacuucgagaagccuaguaacgccaugaacguugaggaguguuucgcucagcacuucccccguguaguucaggucgaugagucaccgcguuccccacgggugaccguggcgguggcugcguuggcggccugccuguggggcaacccgcaggacgcucugauacagacauggugugaagagccuauugagcuaguugguaguccuccggccccugaaugcggcuaauccuaacugcggagcacacguucgcaagccagcgaguggugugucguaacgggcaacucugcagcggaaccgacuacuuuggguguccguguuuccuuuuauucuuaccuuggcugcuuauggugacaauugaaagauuguuaccauauagcuauuggauuggccauccagugucaaauagagcaaucauauaucuguuuguugguuucguucccuuggacuacagaaaucuuaaaacucuuuauuucauauugagacucaauacgauaaaaugggagcacaggugucaacacaaaagacaggggcacacgagacuaguuugagcgccaguggaaacucaauuauucauuacaccaacauaaacuauuacaaggaugcugcuucaaauucggccaauaggcaagauuuuacacaagacccuaguaaauucacagaaccgguaaaggaugugaugauaaagucgcugccagcgcucaauuccccgacuguagaggagugcggauauagcgacagaguuagaucaauaacacucgggaacucgacuauaacgacacaagagugugcaaacgucguggugggguauggcgucuggcccgauuaucuuagcgacgaagaggcaacagcggaagaccagcccacccaaccugauguggcaacguguagguuuuacacguugaauucagugaaaugggagaugcagucagcgggguggugguggaaguucccagaugcauugucagaaauggggcucuuugggcagaauaugcaauaucacuaccuaggcagaucaggguacacaauucaugugcaaugcaacgcauccaaauuccaccaagguugucugcuuguggugugugugccugaggcugagaugggauguaccaaugcaaaaaacgcacccgcguaugaugaguuguguggaggagagacagcaaagaguuucgaacagaaugcagccacagguaagacagcugugcagacggcugugugcaaugcggguauggguguggggguugguaacuugacuauauacccucaccaauggauuaauuuaagaacaaacaauagugccaccauagugaugccauacauuaauagcgucccaauggacaacauguucaggcauaauaacuuuacauuaaugauaauacccuuugcaccguuggacuacguuacgggagcguccucuuacaucccuaucacagugacaguugccccuaugagcgcugaguacaaugguuugcgucuagcuggucaucaaggcuuaccaacuaugcuuacaccaggcagcacgcaguuuuugacgucagaugauuuucaaucaccaucagcuaugccacaguuugaugugaccccagagaugaacauuccagggcaagugaggaaccugauggaaauugcggaaguugauucugugguaccaaucaauaacuugaaagcuaaucugaugacgauggaggcuuaccgggugcagguuagguccacugacgagaugggaggacagauauuuggcuuccccuuacagccaggggcaucaagcguguuacaaagaacacuacugggagagauauuaaauuacuacacucauuggucagggagccucaaguuaacauuuguguucugugggucggcaauggcaacuggcaaauucuuacuagcauacucaccaccuggagcaggggcaccagacagcaggaagaacgcuauguuagggacccacgucauaugggacguuggacugcaauccagcugugugcucuguguaccguggaucagccagacgcacuacagguauguuguugaugacaaguacacggcuagugguuucauuucgugcugguaccaaacuaaugucauagucccagcugaagcucagaaaucgugcuacauaaugugcuuugugucagcaugcaacgauuucucuguacgcauguugagggacacgcaauucauuaagcaaacaaacuuuuaucagggaccaacagaagaguccguggagagagcaauggggagaguugcagacacgauugcccgcggcccaucgaacucugagcaaaucccagcucugacagcuguggagacuggacauacuucccagguggauccaagugacacgaugcaaacaagacaugugcauaacuaccacuccagaucagaaucaucuauagaaaacuuccugugcagaucugcuugcguaauuuauauaaaauacuccagugcugaaucaaacaaccugaagcgguaugcggaguggguuaucaacacaaggcagguggcucaacuaaggcgaaagauggaaauguucacuuauauucggugcgacauggagcuuaccuuugugaucaccagccaucaggagauguccaccgccacuaacucagauguuccagugcagacacaccaaauaauguacgugccaccuggcggcccuguaccaacgucagucaacgacuacguguggcaaacauccaccaaccccagcaucuuuuggacagagggcaaugcaccaccaaggauguccauaccguucaugaguauuggcaaugcuuacaccauguuuuaugauggguggucaaacuucuccagagacggcauauauggauauaauucauuaaacaacauggggaccauauaugcgcgccauguuaaugauucuagcccagggggacugaccagcaccauccgcaucuacuucaaacccaaacacgucaaagcauaugugccacgccccccccguuugugucaauacaagaaagccaagagugugaacuuugauguugaggccguuacagcggagcgugcaagcuugauaaccacaggccccuauggacaucaaucaggggccguguaugugggcaauuacaagguagucaauaggcacuuggccacgcacguggauuggcaaaauugcgugugggaggauuauaauagagaccuucuagugaguacuaccacggcccacgggugcgacaccauugccagaugccaaugcacaacagguguguacuuuugcgccuccaagagcaaacacuacccaguuagcuuugaaggaccagguuugguggaaguccaagaaagugaauauuacccaaaaagauaccagucucauguguugcuugcuacaggguucuccgaaccaggagauugcgguggaauucucaggugcgaacacggcgucaucggucuugucaccauggguggugaaggcgugguugguuucgccgauguccgugaccuguugugguuggaagaugaugcaauggaacagggagugaaagauuacguugagcaacuugguaaugcuuuuggcucaggauucaccaaccagauaugcgagcagguuaaccuccuaaaagaaucacuaguaggucaggacucaaucuuggagaagucacucaaagcccuaguuaagaucaucucugcccuggugauuguaguaaggaaucaugaugaccugaucacaguuacagcuacacucgcccuuauuggcugcaccucgucuccguggcgauggcuuaagcacaaggugucccaauauuacggaauacccauggcugaacgccagaacaacggguggcuaaagaaauuuacagagaugacuaacgcaugcaaagggauggaguggauagccgucaagauucagaaauuuauagaauggcucaagguuaagauuuugccagaagucaaggaaaagcaugaauuccuaaguagacucaaacagcucccacucuuggagagucagauugccaccauugaacaaagugcacccucucaaagugaccaggaacagcuguucucaaauguccaguacuucgcucacuauugcagaaaguaugcaccgcucuacgcugcagaagccaagaggguguuuucccuugaaaaaaaaaugagcaauuacauacaguucaaguccaaaugccguauugaaccuguaugcuugcuuuugcaugguagcccaggagcggggaagucaguugcuaccaacuuaauugggcggucauuagcugaaaaguuaaacaguucaguguacuccuuaccaccagacccagaccauuucgauggcuacaaacaacaagccgucguaauuauggacgaucuaugccaaaacccggauggcaaggacgugucuuuguucugccaaauggugucuaguguagacuuuguaccaccaauggcugcacuggaggaaaaagguaucuuguucaccuccccuuuuguccuggccucaaccaaugcuggguccaucaacgcgccgacagucucagacagccgggcucuggcaagaagguuccauuuugacaugaauauugaaguuaucuccauguacagccagaauggcaagaucaacaugcccaugucagucaagacgugugaugaagaguguugcccagucaauuucaagagaugcugcccucuaguguguggaaaggcuauccaguuuaucgauagaaagacucaagugagguacucccuagauaugcuagucacggagauguuuagggaauacaaccacaggcacagugucggggcgacccuugaggcacuauuccaaggcccgccaguguacagagaaauuaaaauuagugucacaccugaaaccccaccaccaccaguaaucgcagacuuguugaagucaguggacagcgaggcuguuagagaguacuguaaggagaagggauggcuaguuccugagaucgauucuacucuccaaauugagaagcauaucaguagagcauuuaucugccuccaagcacugacaacuuucgugucuguggccggaauuauuuauaucauuuacaaacuguuugcaggguuccagggugcauauacggggaugccuaaccaaaaaccuaaagugccuacacuaaggcaggcuaaagugcagggucccgcuuuugaguucgcuguggccaugaugaagaggaacuccaguacggugaaaacagaguauggcgaguucaccauguuaggcaucuaugacaggugggcuguccuaccacgccacgcuaaacccgggccgacuauucuuaugaaugaccaggaggucggugugcuggaugccaaggaacuaauagacagagaugguacaaaucuggagcugacacuacugaaacucaaccggaaugagaaauucagggacaucagagguuuucuagccaaggaggaaguggagguuaaugaagcuguccuagcaaucaacacuagcaaauuucccaacauguacauccccguagggcaggucacagacuauggcuuccuaaaccuaggugguacucccacaaagagaaugcucauguacaacuucccuacaagggcuggacaguguggcgguguucucauguccacuggcaaggugcuagggauccacguuggugggaauggucaccaggguuucucagcagcgcuccuuaagcacuacuuuaaugaugagcagggggagaucgaguucaucgaaagcucgaaagacgcagguuucccagucaucaauacaccaaguagaacuaagcuagaaccaagcgucuuccaucacgucuuugaaggaaacaaggaaccagcaguccucaggaacggcgacccgcgccuuaaagucaacuuugaggaggcuauauuuuccaaauacauaggaaacgucaacacacauguggacgaguacaugcuagaagcuguggaucacuaugcagggcaauuggccacucuugacauuaacacugagccaaugaaacuggaagaugcaguguacggcacggaagggcuagaggcucuugauuuaacaacaagugcgggguacccauauguugcauuaggcauuaagaagagggacauccuauccaaaaagaccaaagaccugaccaaauugaaggaauguauggacaaguacggauuaaacuugccgauggugacauacgugaaggaugagcuuagaucagcagagaagguggccaaagggaaaucuagacucauugaagcauccagcuugaacgacucuguugcgaugaggcaaacauuugguaauuuguacaaggcauuccacuuaaacccggggguuguaacgggcagugcagucgggugcgauccagacguuuucuggaguaaaauaccugugaugcuagacggacaccuuauagccuucgacuacuccgguuaugacgccagucugagccccgugugguuugcuugucuaaaguugcugcuugaaaaacucggguacacacauaaagagacaaacuacauugacuacuuaugcaacucccaccaccuauacagagacaaacacuacuuuguacguggcgguaugcccucagggugcucugguaccagcaucuucaacucaaugaucaauaacaucauuaucaggaccuuaauguugaagguguacaaagguauugacuuggaucaauucaggaugauugcauauggugaugaugugauugcaucauauccuuggcccauagacgccucucugcucgcugaagcugguaaagacuacgggcuaaucaugacaccagcggauaaaggagaguguuuuaacgaagucaccuggacuaaugucaccuuucuaaagagguauuuuagagcagaugaacaauacccuuucuugguucacccagugaugcccaugaaagacauccacgagucuaucagguggaccaaagauccaaagaacacucaagaucaugugcgcucccugugcuuauuggcuuggcacaauggagagcacgaauaugaggaguucauccaaaagaucagaagcgucccaguugggcgcugcuugacucugcccgcguuuucgacccuacguaggaaaugguuggauuccuuuuaaauuagagacaauuugaaacaauuuaaauuggcuuaacccuacugcacuaaccgaacuagauaacggugcaguagggguaaauucuccgcauucggugcgg

SEQ ID NO: 2 - protein, 2183 aa, Coxsackie B4 virus
MGAQVSTQKTGAHETSLSASGNSIIHYTNINYYKDAASNSANRQDFTQDPSKFTEPVKDVMIKSLPALNSPTVEECGYSDRVRSITLGNSTITTQECANVVVGYGVWPDYLSDEEATAEDQPTQPDVATCRFYTLNSVKWEMQSAGWWWKFPDALSEMGLFGQNMQYHYLGRSGYTIHVQCNASKFHQGCLLVVCVPEAEMGCTNAKNAPAYDELCGGETAKSFEQNAATGKTAVQTAVCNAGMGVGVGNLTIYPHQWINLRTNNSATIVMPYINSVPMDNMFRHNNFTLMIIPFAPLDYVTGASSYIPITVTVAPMSAEYNGLRLAGHQGLPTMLTPGSTQFLTSDDFQSPSAMPQFDVTPEMNIPGQVRNLMEIAEVDSVVPINNLKANLMTMEAYRVQVRSTDEMGGQIFGFPLQPGASSVLQRTLLGEILNYYTHWSGSLKLTFVFCGSAMATGKFLLAYSPPGAGAPDSRKNAMLGTHVIWDVGLQSSCVLCVPWISQTHYRYVVDDKYTASGFISCWYQTNVIVPAEAQKSCYIMCFVSACNDFSVRMLRDTQFIKQTNFYQGPTEESVERAMGRVADTIARGPSNSEQIPALTAVETGHTSQVDPSDTMQTRHVHNYHSRSESSIENFLCRSACVIYIKYSSAESNNLKRYAEWVINTRQVAQLRRKMEMFTYIRCDMELTFVITSHQEMSTATNSDVPVQTHQIMYVPPGGPVPTSVNDYVWQTSTNPSIFWTEGNAPPRMSIPFMSIGNAYTMFYDGWSNFSRDGIYGYNSLNNMGTIYARHVNDSSPGGLTSTIRIYFKPKHVKAYVPRPPRLCQYKKAKSVNFDVEAVTAERASLITTGPYGHQSGAVYVGNYKVVNRHLATHVDWQNCVWEDYNRDLLVSTTTAHGCDTIARCQCTTGVYFCASKSKHYPVSFEGPGLVEVQESEYYPKRYQSHVLLATGFSEPGDCGGILRCEHGVIGLVTMGGEGVVGFADVRDLLWLEDDAMEQGVKDYVEQLGNAFGSGFTNQICEQVNLLKESLVGQDSILEKSLKALVKIISALVIVVRNHDDLITVTATLALIGCTSSPWRWLKHKVSQYYGIPMAERQNNGWLKKFTEMTNACKGMEWIAVKIQKFIEWLKVKILPEVKEKHEFLSRLKQLPLLESQIATIEQSAPSQSDQEQLFSNVQYFAHYCRKYAPLYAAEAKRVFSLEKKMSNYIQFKSKCRIEPVCLLLHGSPGAGKSVATNLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRKTQVRYSLDMLVTEMFREYNHRHSVGATLEALFQGPPVYREIKISVTPETPPPPVIADLLKSVDSEAVREYCKEKGWLVPEIDSTLQIEKHISRAFICLQALTTFVSVAGIIYIIYKLFAGFQGAYTGMPNQKPKVPTLRQAKVQGPAFEFAVAMMKRNSSTVKTEYGEFTMLGIYDRWAVLPRHAKPGPTILMNDQEVGVLDAKELIDRDGTNLELTLLKLNRNEKFRDIRGFLAKEEVEVNEAVLAINTSKFPNMYIPVGQVTDYGFLNLGGTPTKRMLMYNFPTRAGQCGGVLMSTGKVLGIHVGGNGHQGFSAALLKHYFNDEQGEIEFIESSKDAGFPVINTPSRTKLEPSVFHHVFEGNKEPAVLRNGDPRLKVNFEEAIFSKYIGNVNTHVDEYMLEAVDHYAGQLATLDINTEPMKLEDAVYGTEGLEALDLTTSAGYPYVALGIKKRDILSKKTKDLTKLKECMDKYGLNLPMVTYVKDELRSAEKVAKGKSRLIEASSLNDSVAMRQTFGNLYKAFHLNPGVVTGSAVGCDPDVFWSKIPVMLDGHLIAFDYSGYDASLSPVWFACLKLLLEKLGYTHKETNYIDYLCNSHHLYRDKHYFVRGGMPSGCSGTSIFNSMINNIIIRTLMLKVYKGIDLDQFRMIAYGDDVIASYPWPIDASLLAEAGKDYGLIMTPADKGECFNEVTWTNVTFLKRYFRADEQYPFLVHPVMPMKDIHESIRWTKDPKNTQDHVRSLCLLAWHNGEHEYEEFIQKIRSVPVGRCLTLPAFSTLRRKWLDSF

SEQ ID NO: 3 - protein, 69 aa, Artificial Sequence
  Fragment derived from coxsackie B4 virus (CBV4)
MGAQVSTQKTGAHETSLSASGNSIIHYTNINYYKDAASNSANRQDFTQDPSKFTEPVKDVMIKSLPALN

SEQ ID NO: 4 - protein, 261 aa, Artificial Sequence
  Fragment derived from coxsackie B4 virus (CBV4)
SPTVEECGYSDRVRSITLGNSTITTQECANVVVGYGVWPDYLSDEEATAEDQPTQPDVATCRFYTLNSVKWEMQSAGWWWKFPDALSEMGLFGQNMQYHYLGRSGYTIHVQCNASKFHQGCLLVVCVPEAEMGCTNAKNAPAYDELCGGETAKSFEQNAATGKTAVQTAVCNAGMGVGVGNLTIYPHQWINLRTNNSATIVMPYINSVPMDNMFRHNNFTLMIIPFAPLDYVTGASSYIPITVTVAPMSAEYNGLRLAGHQ

SEQ ID NO: 5 - protein, 238 aa, Artificial Sequence
  Fragment derived from coxsackie B4 virus (CBV4)
GLPTMLTPGSTQFLTSDDFQSPSAMPQFDVTPEMNIPGQVRNLMEIAEVDSVVPINNLKANLMTMEAYRVQVRSTDEMGGQIFGFPLQPGASSVLQRTLLGEILNYYTHWSGSLKLTFVFCGSAMATGKFLLAYSPPGAGAPDSRKNAMLGTHVIWDVGLQSSCVLCVPWISQTHYRYVVDDKYTASGFISCWYQTNVIVPAEAQKSCYIMCFVSACNDFSVRMLRDTQFIKQTNFYQ

SEQ ID NO: 6 - protein, 281 aa, Artificial Sequence
  Fragment derived from coxsackie B4 virus (CBV4)
GPTEESVERAMGRVADTIARGPSNSEQIPALTAVETGHTSQVDPSDTMQTRHVHNYHSRSESSIENFLCRSACVIYIKYSSAESNNLKRYAEWVINTRQVAQLRRKMEMFTYIRCDMELTFVITSHQEMSTATNSDVPVQTHQIMYVPPGGPVPTSVNDYVWQTSTNPSIFWTEGNAPPRMSIPFMSIGNAYTMFYDGWSNFSRDGIYGYNSLNNMGTIYARHVNDSSPGGLTSTIRIYFKPKHVKAYVPRPPRLCQYKKAKSVNFDVEAVTAERASLITT

SEQ ID NO: 7 - protein, 150 aa, Artificial Sequence
  Fragment derived from coxsackie B4 virus (CBV4)-2A region encoding viral proteases
GPYGHQSGAVYVGNYKVVNRHLATHVDWQNCVWEDYNRDLLVSTTTAHGCDTIARCQCTTGVYFCASKSKHYPVSFEGPGLVEVQESEYYPKRYQSHVLLATGFSEPGDCGGILRCEHGVIGLVTMGGEGVVGFADVRDLLWLEDDAMEQ

SEQ ID NO: 8 - protein, 99 aa, Artificial Sequence
  Fragment derived from coxsackie B4 virus (CBV4)
GVKDYVEQLGNAFGSGFTNQICEQVNLLKESLVGQDSILEKSLKALVKIISALVIVVRNHDDLITVTATLALIGCTSSPWRWLKHKVSQYYGIPMAERQ

SEQ ID NO: 9 - protein, 329 aa, Artificial Sequence
  Fragment derived from coxsackie B4 virus (CBV4)-2C region encoding the viral RNA helicase
NNGWLKKFTEMTNACKGMEWIAVKIQKFIEWLKVKILPEVKEKHEFLSRLKQLPLLESQIATIEQSAPSQSDQEQLFSNVQYFAHYCRKYAPLYAAEAKRVFSLEKKMSNYIQFKSKCRIEPVCLLLHGSPGAGKSVATNLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRKTQVRYSLDMLVTEMFREYNHRHSVGATLEALFQ

SEQ ID NO: 10 - protein, 89 aa, Artificial Sequence
  Fragment derived from coxsackie B4 virus (CBV4)
GPPVYREIKISVTPETPPPPVIADLLKSVDSEAVREYCKEKGWLVPEIDSTLQIEKHISRAFICLQALTTFVSVAGIIYIIYKLFAGFQ

SEQ ID NO: 11 - protein, 22 aa, Artificial Sequence
  Fragment derived from coxsackie B4 virus (CBV4)-VPg protein
GAYTGMPNQKPKVPTLRQAKVQ

SEQ ID NO: 12 - protein, 183 aa, Artificial Sequence
  Fragment derived from coxsackie B4 virus (CBV4)-3C region encoding viral proteases
GPAFEFAVAMMKRNSSTVKTEYGEFTMLGIYDRWAVLPRHAKPGPTILMNDQEVGVLDAKELIDRDGTNLELTLLKLNRNEKFRDIRGFLAKEEVEVNEAVLAINTSKFPNMYIPVGQVTDYGFLNLGGTPTKRMLMYNFPTRAGQCGGVLMSTGKVLGIHVGGNGHQGFSAALLKHYFNDEQ

SEQ ID NO: 13 - protein, 462 aa, Artificial Sequence
  Fragment derived from coxsackie B4 virus (CBV4)-3D region encoding viral polymerase
GEIEFIESSKDAGFPVINTPSRTKLEPSVFHHVFEGNKEPAVLRNGDPRLKVNFEEAIFSKYIGNVNTHVDEYMLEAVDHYAGQLATLDINTEPMKLEDAVYGTEGLEALDLTTSAGYPYVALGIKKRDILSKKTKDLTKLKECMDKYGLNLPMVTYVKDELRSAEKVAKGKSRLIEASSLNDSVAMRQTFGNLYKAFHLNPGVVTGSAVGCDPDVFWSKIPVMLDGHLIAFDYSGYDASLSPVWFACLKLLLEKLGYTHKETNYIDYLCNSHHLYRDKHYFVRGGMPSGCSGTSIFNSMINNIIIRTLMLKVYKGIDLDQFRMIAYGDDVIASYPWPIDASLLAEAGKDYGLIMTPADKGECFNEVTWTNVTFLKRYFRADEQYPFLVHPVMPMKDIHESIRWTKDPKNTQDHVRSLCLLAWHNGEHEYEEFIQKIRSVPVGRCLTLPAFSTLRRKWLDSF

SEQ ID NO: 14 - DNA, 7395 bp, Coxsackie B4 virus
  misc_feature: DNA sequence corresponding to RNA of SEQ ID NO: 1
ttaaaacagcctgtgggttgtacccacccacagggcccaatgggcgctagcacactggtattccggtacctttgtgcgcctgttttataacccccccccagttcgcaacttagaagcaaagaaacaatggtcaatagctgacgcagcaacccagctgtgttttggccaagcacttctgtgtccccggactgagtatcaataagctgcttgcgcggctgaaggagaaaccgttcgttacccggccaactacttcgagaagcctagtaacgccatgaacgttgaggagtgtttcgctcagcacttcccccgtgtagttcaggtcgatgagtcaccgcgttccccacgggtgaccgtggcggtggctgcgttggcggcctgcctgtggggcaacccgcaggacgctctgatacagacatggtgtgaagagcctattgagctagttggtagtcctccggcccctgaatgcggctaatcctaactgcggagcacacgttcgcaagccagcgagtggtgtgtcgtaacgggcaactctgcagcggaaccgactactttgggtgtccgtgtttccttttattcttaccttggctgcttatggtgacaattgaaagattgttaccatatagctattggattggccatccagtgtcaaatagagcaatcatatatctgtttgttggtttcgttcccttggactacagaaatcttaaaactctttatttcatattgagactcaatacgataaaatgggagcacaggtgtcaacacaaaagacaggggcacacgagactagtttgagcgccagtggaaactcaattattcattacaccaacataaactattacaaggatgctgcttcaaattcggccaataggcaagattttacacaagaccctagtaaattcacagaaccggtaaaggatgtgatgataaagtcgctgccagcgctcaattccccgactgtagaggagtgcggatatagcgacagagttagatcaataacactcgggaactcgactataacgacacaagagtgtgcaaacgtcgtggtggggtatggcgtctggcccgattatcttagcgacgaagaggcaacagcggaagaccagcccacccaacctgatgtggcaacgtgtaggttttacacgttgaattcagtgaaatgggagatgcagtcagcggggtggtggtggaagttcccagatgcattgtcagaaatggggctctttgggcagaatatgcaatatcactacctaggcagatcagggtacacaattcatgtgcaatgcaacgcatccaaattccaccaaggttgtctgcttgtggtgtgtgtgcctgaggctgagatgggatgtaccaatgcaaaaaacgcacccgcgtatgatgagttgtgtggaggagagacagcaaagagtttcgaacagaatgcagccacaggtaagacagctgtgcagacggctgtgtgcaatgcgggtatgggtgtgggggttggtaacttgactatataccctcaccaatggattaatttaagaacaaacaatagtgccaccatagtgatgccatacattaatagcgtcccaatggacaacatgttcaggcataataactttacattaatgataataccctttgcaccgttggactacgttacgggagcgtcctcttacatccctatcacagtgacagttgcccctatgagcgctgagtacaatggtttgcgtctagctggtcatcaaggcttaccaactatgcttacaccaggcagcacgcagtttttgacgtcagatgattttcaatcaccatcagctatgccacagtttgatgtgaccccagagatgaacattccagggcaagtgaggaacctgatggaaattgcggaagttgattctgtggtaccaatcaataacttgaaagctaatctgatgacgatggaggcttaccgggtgcaggttaggtccactgacgagatgggaggacagatatttggcttccccttacagccaggggcatcaagcgtgttacaaagaacactactgggagagatattaaattactacactcattggtcagggagcctcaagttaacatttgtgttctgtgggtcggcaatggcaactggcaaattcttactagcatactcaccacctggagcaggggcaccagacagcaggaagaacgctatgttagggacccacgtcatatgggacgttggactgcaatccagctgtgtgctctgtgtaccgtggatcagccagacgcactacaggtatgttgttgatgacaagtacacggctagtggtttcatttcgtgctggtaccaaactaatgtcatagtcccagctgaagctcagaaatcgtgctacataatgtgctttgtgtcagcatgcaacgatttctctgtacgcatgttgagggacacgcaattcattaagcaaacaaacttttatcagggaccaacagaagagtccgtggagagagcaatggggagagttgcagacacgattgcccgcggcccatcgaactctgagcaaatcccagctctgacagctgtggagactggacatacttcccaggtggatccaagtgacacgatgcaaacaagacatgtgcataactaccactccagatcagaatcatctatagaaaacttcctgtgcagatctgcttgcgtaatttatataaaatactccagtgctgaatcaaacaacctgaagcggtatgcggagtgggttatcaacacaaggcaggtggctcaactaaggcgaaagatggaaatgttcacttatattcggtgcgacatggagcttacctttgtgatcaccagccatcaggagatgtccaccgccactaactcagatgttccagtgcagacacaccaaataatgtacgtgccacctggcggccctgtaccaacgtcagtcaacgactacgtgtggcaaacatccaccaaccccagcatcttttggacagagggcaatgcaccaccaaggatgtccataccgttcatgagtattggcaatgcttacaccatgttttatgatgggtggtcaaacttctccagagacggcatatatggatataattcattaaacaacatggggaccatatatgcgcgccatgttaatgattctagcccagggggactgaccagcaccatccgcatctacttcaaacccaaacacgtcaaagcatatgtgccacgccccccccgtttgtgtcaatacaagaaagccaagagtgtgaactttgatgttgaggccgttacagcggagcgtgcaagcttgataaccacaggcccctatggacatcaatcaggggccgtgtatgtgggcaattacaaggtagtcaataggcacttggccacgcacgtggattggcaaaattgcgtgtgggaggattataatagagaccttctagtgagtactaccacggcccacgggtgcgacaccattgccagatgccaatgcacaacaggtgtgtacttttgcgcctccaagagcaaacactacccagttagctttgaaggaccaggtttggtggaagtccaagaaagtgaatattacccaaaaagataccagtctcatgtgttgcttgctacagggttctccgaaccaggagattgcggtggaattctcaggtgcgaacacggcgtcatcggtcttgtcaccatgggtggtgaaggcgtggttggtttcgccgatgtccgtgacctgttgtggttggaagatgatgcaatggaacagggagtgaaagattacgttgagcaacttggtaatgcttttggctcaggattcaccaaccagatatgcgagcaggttaacctcctaaaagaatcactagtaggtcaggactcaatcttggagaagtcactcaaagccctagttaagatcatctctgccctggtgattgtagtaaggaatcatgatgacctgatcacagttacagctacactcgcccttattggctgcacctcgtctccgtggcgatggcttaagcacaaggtgtcccaatattacggaatacccatggctgaacgccagaacaacgggtggctaaagaaatttacagagatgactaacgcatgcaaagggatggagtggatagccgtcaagattcagaaatttatagaatggctcaaggttaagattttgccagaagtcaaggaaaagcatgaattcctaagtagactcaaacagctcccactcttggagagtcagattgccaccattgaacaaagtgcaccctctcaaagtgaccaggaacagctgttctcaaatgtccagtacttcgctcactattgcagaaagtatgcaccgctctacgctgcagaagccaagagggtgttttcccttgaaaaaaaaatgagcaattacatacagttcaagtccaaatgccgtattgaacctgtatgcttgcttttgcatggtagcccaggagcggggaagtcagttgctaccaacttaattgggcggtcattagctgaaaagttaaacagttcagtgtactccttaccaccagacccagaccatttcgatggctacaaacaacaagccgtcgtaattatggacgatctatgccaaaacccggatggcaaggacgtgtctttgttctgccaaatggtgtctagtgtagactttgtaccaccaatggctgcactggaggaaaaaggtatcttgttcacctccccttttgtcctggcctcaaccaatgctgggtccatcaacgcgccgacagtctcagacagccgggctctggcaagaaggttccattttgacatgaatattgaagttatctccatgtacagccagaatggcaagatcaacatgcccatgtcagtcaagacgtgtgatgaagagtgttgcccagtcaatttcaagagatgctgccctctagtgtgtggaaaggctatccagtttatcgatagaaagactcaagtgaggtactccctagatatgctagtcacggagatgtttagggaatacaaccacaggcacagtgtcggggcgacccttgaggcactattccaaggcccgccagtgtacagagaaattaaaattagtgtcacacctgaaaccccaccaccaccagtaatcgcagacttgttgaagtcagtggacagcgaggctgttagagagtactgtaaggagaagggatggctagttcctgagatcgattctactctccaaattgagaagcatatcagtagagcatttatctgcctccaagcactgacaactttcgtgtctgtggccggaattatttatatcatttacaaactgtttgcagggttccagggtgcatatacggggatgcctaaccaaaaacctaaagtgcctacactaaggcaggctaaagtgcagggtcccgcttttgagttcgctgtggccatgatgaagaggaactccagtacggtgaaaacagagtatggcgagttcaccatgttaggcatctatgacaggtgggctgtcctaccacgccacgctaaacccgggccgactattcttatgaatgaccaggaggtcggtgtgctggatgccaaggaactaatagacagagatggtacaaatctggagctgacactactgaaactcaaccggaatgagaaattcagggacatcagaggttttctagccaaggaggaagtggaggttaatgaagctgtcctagcaatcaacactagcaaatttcccaacatgtacatccccgtagggcaggtcacagactatggcttcctaaacctaggtggtactcccacaaagagaatgctcatgtacaacttccctacaagggctggacagtgtggcggtgttctcatgtccactggcaaggtgctagggatccacgttggtgggaatggtcaccagggtttctcagcagcgctccttaagcactactttaatgatgagcagggggagatcgagttcatcgaaagctcgaaagacgcaggtttcccagtcatcaatacaccaagtagaactaagctagaaccaagcgtcttccatcacgtctttgaaggaaacaaggaaccagcagtcctcaggaacggcgacccgcgccttaaagtcaactttgaggaggctatattttccaaatacataggaaacgtcaacacacatgtggacgagtacatgctagaagctgtggatcactatgcagggcaattggccactcttgacattaacactgagccaatgaaactggaagatgcagtgtacggcacggaagggctagaggctcttgatttaacaacaagtgcggggtacccatatgttgcattaggcattaagaagagggacatcctatccaaaaagaccaaagacctgaccaaattgaaggaatgtatggacaagtacggattaaacttgccgatggtgacatacgtgaaggatgagcttagatcagcagagaaggtggccaaagggaaatctagactcattgaagcatccagcttgaacgactctgttgcgatgaggcaaacatttggtaatttgtacaaggcattccacttaaacccgggggttgtaacgggcagtgcagtcgggtgcgatccagacgttttctggagtaaaatacctgtgatgctagacggacaccttatagccttcgactactccggttatgacgccagtctgagccccgtgtggtttgcttgtctaaagttgctgcttgaaaaactcgggtacacacataaagagacaaactacattgactacttatgcaactcccaccacctatacagagacaaacactactttgtacgtggcggtatgccctcagggtgctctggtaccagcatcttcaactcaatgatcaataacatcattatcaggaccttaatgttgaaggtgtacaaaggtattgacttggatcaattcaggatgattgcatatggtgatgatgtgattgcatcatatccttggcccatagacgcctctctgctcgctgaagctggtaaagactacgggctaatcatgacaccagcggataaaggagagtgttttaacgaagtcacctggactaatgtcacctttctaaagaggtattttagagcagatgaacaataccctttcttggttcacccagtgatgcccatgaaagacatccacgagtctatcaggtggaccaaagatccaaagaacactcaagatcatgtgcgctccctgtgcttattggcttggcacaatggagagcacgaatatgaggagttcatccaaaagatcagaagcgtcccagttgggcgctgcttgactctgcccgcgttttcgaccctacgtaggaaatggttggattccttttaaattagagacaatttgaaacaatttaaattggcttaaccctactgcactaaccgaactagataacggtgcagtaggggtaaattctccgcattcggtgcgg

SEQ ID NO: 15 - RNA, 7395 nt, Coxsackie B4 virus
  misc_feature: Prototype strain of coxsackie B4 virus: JVB
uuaaaacagccuguggguuguacccacccacagggcccaaugggcgcuagcacacugguauuccgguaccuuugugcgccuguuuuauaacccccccccaguucgcaacuuagaagcaaagaaacaauggucaauuacugacgcagcaacccagcuguguuuuggccaaguacuucuguguccccggacugaguaucaauaagcugcuugcgcggcugaaggagaaaccguucguuacccggccaacuacuucgagaagccuaguaacgccaugaacguugaggaguguuucgcucagcacuucccccguguaguucaggucgaugagucaccgcguuccccacgggugaccguggcgguggcugcguuggcggccugccuguggggcaacccgcaggacgcucugauacagacauggugugaagagccuauugagcuaguugguaguccuccggccccugaaugcggcuaauccuaacugcggagcacacguucgcaagccagcgaguggugugucguaacgggcaacucugcagcggaaccgaguacuuuggguguccguguuuccuuuuauucuuaccuuggcugcuuauggugacaauugaaagauuguuaccauauagcuauuggauuggccauccagugucaaauagagcaaucauauaucuguuuguugguuucguucccuuggacuacagaaaucuuaaaacucuuuauuucauauugagacucaauacgauaaaaugggagcacaggugucaacacaaaagacaggggcacacgagacuaguuugagcgccaguggaaacucgauuauucauuacaccaacauaaacuauuacaaggaugcugcuucaaauucggccaauaggcaagauuuuacacaagacccuaguaaauucacagaaccgguaaaggaugugaugauaaagucgcugccagcgcucaauuccccgacuguagaggagugcggauauagcgacagaguuagaucaauaacacucgggaacucgacuauaacgacacaagagugugcaaacgucguggugggguauggcgucuggcccgauuaucuuagcgacgaagaggcaacagcggaagaccagcccacccaaccugauguggcaacguguagguuuuacacguugaauucagugaaaugggagaugcagucagcgggguggugguggaaguucccagaugcauugucagaaauggggcucuuugggcagaauaugcaauaucacuaccuaggcagaucaggguacacaauucaugugcaaugcaacgcauccaaauuccaccaagguugucugcuuguggugugugugccugaggcugagaugggauguaccaaugcagaaaacgcacccgcguauggugauuuguguggaggagagacagcaaagaguuucgaacagaaugcagccacagguaagacagcugugcagacggcugugugcaaugcggguauggguguggggguugguaacuugacuauauacccucaccaauggauuaauuuaagaacaaacaauagugccaccauagugaugccauacauuaauagcgucccaauggacaacauguucaggcauaauaacuuuacauuaaugauaauacccuuugcaccguuggacuacguuacgggagcguccucuuacaucccuaucacagugacaguugccccuaugagcgcugaguacaaugguuugcgucuagcuggucaucaaggcuuaccaacuaugcuuacaccaggcagcacgcaguuuuugacgucagaugauuuucaaucaccaucagcuaugccacaguuugaugugaccccagagaugaacauuccagggcaagugaggaaccugauggaaauugcggaaguugauucugugguaccaaucaauaacuugaaagcuaaucugaugacgauggaggcuuaccgggugcagguuagguccacugacgagaugggaggacagauauuuggcuuccccuuacagccaggggcaucaagcguguuacaaagaacacuacugggagagauauuaaauuacuacacucauuggucagggagccucaaguuaacauuuguguucugugggucggcaauggcaacuggcaaauucuuacuagcauacucaccaccuggagcaggggcaccagacagcaggaagaacgcuauguuagggacccacgucauaugggacguuggacugcaauccagcugugugcucuguguaccguggaucagccagacgcacuacagguauguuguugaugacaaguacacggcuagugguuucauuucgugcugguaccaaacuaaugucauagucccagcugaagcucagaaaucgugcuacauaaugugcuuugugucagcaugcaacgauuucucuguacgcauguugagggacacgcaauucauuaagcaaacaaacuuuuaucagggaccaacagaagaguccguggagagagcaauggggagaguugcagacacgauugcccgcggcccaucgaacucugagcaaaucccagcucugacagcuguggagacuggacauacuucccagguggauccaagugacacgaugcaaacaagacaugugcauaacuaccacuccagaucagaaucaucuauagaaaacuuccugugcagaucugcuugcguaauuuauauaaaauacuccagugcugaaucaaacaaccugaagcgguaugcggaguggguuaucaacacaaggcagguggcucaacuaaggcgaaagauggaaauguucacuuauauucggugcgacauggagcuuaccuuugugauuaccagccaucaggagauguccaccgccacuaacucagauguuccagugcagacacaccaaauaauguacgugccaccuggcggcccuguaccaacgucagucaacgacuacguguggcaaacauccaccaaccccagcaucuuuuggacagagggcaaugcaccaccaaggauguccauaccguucaugaguauuggcaaugcuuacaccauguuuuaugacggguggucaaacuucuccagagacggcauauauggauauaauucauuaaacaacauggggaccauauaugcgcgccauguuaaugauucuagcccagggggacugaccagcaccauccgcaucuacuucaaacccaaacacgucaaagcauaugugccacgccccccccguuugugucaauacaagaaagccaagagugugaacuuugauguugaggccguuacagcggagcgugcaagcuugauaaccacaggccccuauggacaucaaucaggggccguguaugugggcaauuacaagguagucaauaggcacuuggccacgcacguggauuggcaaaauugcgugugggaggauuauaauagagaccuucuagugaguacuaccacggcccacgggugcgacaccauugccagaugccaaugcacaacagguguguacuuuugcgccuccaagagcaaacacuacccaguuagcuuugaaggaccagguuugguggaaguccaagaaagugaauauuacccaaaaagauaccagucucauguguugcuugcuacaggguucuccgaaccaggagauugcgguggaauucucaggugcgaacacggcgucaucggucuugucaccauggguggugaaggcgugguugguuucgccgauguccgugaccuguugugguuggaagaugaugcaauggaacagggagugaaagauuacguugagcaacuugguaaugcuuuuggcucaggauucaccaaccagauaugcgagcagguuaaccuccuaaaagaaucacuaguaggucaggacucaaucuuggagaagucacucaaagcccuaguuaagaucaucucugcccuggugauuguaguaaggaaucaugaugaccugaucacaguuacagcuacacucgcccuuauuggcugcaccucgucuccguggcgauggcuuaagcacaaggugucccaauauuacggaauacccauggcugaacggcagaacaacggguggcuaaagaaauuuacagagaugacuaacgcaugcaaagggauggaguggauagccgucaagauucagaaauuuauagaauggcucaagguuaagauuuugccagaagucaaggaaaagcaugaauuccuaaguagacucaaacagcucccacucuuggagagucagauugccaccauugaacaaagugcacccucucaaagugaccaggaacagcuguucucaaauguccaguacuucgcucacuauugcagaaaguacgcaccgcucuacgcugcagaagccaagaggguguuuucccuugaaaaaaaaaugagcaauuacauacaguucaaguccaaaugccguauugaaccuguaugcuugcuuuugcaugguagcccaggagcggggaagucaguugcuaccaacuuaauugggcggucauuagcugaaaaguuaaacaguucaguguacuccuuaccaccagacccagaccauuucgauggcuacaaacaacaagccgucguaauuauggacgaucuaugccaaaacccggauggcaaggacgugucuuuguucugccaaauggugucuaguguagacuuuguaccaccaauggcugcacuggaggaaaaagguaucuuguucaccuccccuuuuguccuggccucaaccaaugcuggguccaucaacgcgccgacagucucagacagccgggcucuggcaagaagguuccauuuugacaugaauauugaaguuaucuccauguacagccagaauggcaagaucaacaugcccaugucagucaagacgugugaugaagaguguugcccagucaauuucaagagaugcugcccucuaguguguggaaaggcuauccaguuuaucgauagaaagacucaagugagguacucccuagauaugcuagucacggagauguuuagggaauacaaccacaggcacagugucggggcgacccuugaggcgcuauuccaaggcccgccaguguacagagaaauuaaaauuagugucacaccugaaaccccaccaccaccaguaaucgcagacuuguugaagucaguggacaggcaggcuauuagagaguacuguaaggagaagggauggcuaguuccugagaucgauucuauucuccaaauugagaagcaugucaguagagcauuuaucugccuccaagcacugacaacuuucgugucuguggccggaauuauuuauaucauuuacaaacuguuugcaggguuccagggugcauauacggggaugccuaaccaaaaaccuaaagugccuacacuaaggcaggcuaaagugcagggucccgcuuuugaguucgcuguggccaugaugaagaggaacuccaguacggugaaaacagaguauggcgaguucaccauguuaggcaucuaugacaggugggcuguccuaccacgccacgcuaaacccgggccgacuauucuuaugaaugaccaggaggucggugugcuggaugccaaggaauuaauagacagagaugguacaaaucuggagcugacacuacugaaacucaaccggaaugagaaauucagggacaucagagguuuucuagccaaggaggaaguggagguuaaugaagcuguccuagcaaucaacacuagcaaauuuccaaacauguacauccccguaggcagggucacagacuauggcuuccuaaaccuaggugguacucccacaaagagaaugcucauguacaacuucccuacaagggcuggacaguguggcgguguucucauguccacuggcaaggugcuagggauccacguuggugggaauggucaccaggguuucucagcaggccuccuuaagcacuacuuuaaugaugagcagggggagaucgaguucaucgaaagcucgaaagaugcagguuucccagucaucaauacaccaaguagaacuaagcuagaaccaagcgucuuccaucacgucuuugaaggaaacaaggaaccagcaguccucaggaacggcgacccgcgccuuaaagucaacuuugaggaggcuauauuuuucaaauacauaggaaacgucaacacacauguggacgaguacaugcuagaagcuguggaucacuaugcagggcaauuggccacucuugacauuaacacugagccaaugaaacuggaagaugcaguguacggcacggaagggcuagaggcucuugauuuaacaacaagugcgggguacccauauguugcauuaggcauuaagaagagggacauccuauccaaaaagaccaaagaccugaccaaauugaaggaauguauggacaaguacggauuaaacuugccgauggugacauacgugaaggaugagcuuagaucagcagagaagguggccaaagggaaaucuagacucauugaagcauccagcuugaacgacucuguugcgaugaggcaaacauuugguaauuuguacaaggcauuccacuuaaacccggggauuguaacgggcagugcagucgggugcgauccagacguuuucuggaguaaaauaccugugaugcuagacggacaccuuauagccuucgacuacuccgguuaugacgccagucugagccccgugugguuugcuugucuaaaguugcugcuugaaaaacucggguacacacauaaagagacaaacuacauugacuacuuaugcaacucccaccaccuauacagagacaaacacuacuuuguacguggcgguaugcccucagggugcucugguaccagcaucuucaacucaaugaucaauaacaucauuaucaggaccuuaauguugaagguguacaaagguauugacuuggaucaauucaggaugauugcauauggugaugaugugauugcaucauauccuuggcccauagacgccucucugcucgcugaagcugguaaagacuacgggcuaaucaugacaccagcggauaaaggagaguguuuuaacgaagucaccuggacuaaugucaccuuucuaaagagguauuuuagagcagaugaacaauacccuuucuugguucacccagugaugcccaugaaagacauccacgagucuaucagguggaccaaagauccaaagaacacucaagaucaugugcgcucccugugcuuauuggcuuggcacaauggagagcacgaauaugaggaguucauccaaaagaucagaagcgucccaguugggcgcugcuugacucugcccgcguuuucgacccuacguaggaaaugguuggauuccuuuuaaauuagagacaauuugaaacaauuuaaauuggcuuaacccuacugcacuaaccgaacuagauaacggugcaguagggguaaauucuccgcguucggugcgg

SEQ ID NO: 16 - protein, 2183 aa, Coxsackie B4 virus
  misc_feature: Prototype strain of coxsackie B4 virus: JVB
MGAQVSTQKTGAHETSLSASGNSIIHYTNINYYKDAASNSANRQDFTQDPSKFTEPVKDVMIKSLPALNSPTVEECGYSDRVRSITLGNSTITTQECANVVVGYGVWPDYLSDEEATAEDQPTQPDVATCRFYTLNSVKWEMQSAGWWWKFPDALSEMGLFGQNMQYHYLGRSGYTIHVQCNASKFHQGCLLVVCVPEAEMGCTNAENAPAYGDLCGGETAKSFEQNAATGKTAVQTAVCNAGMGVGVGNLTIYPHQWINLRTNNSATIVMPYINSVPMDNMFRHNNFTLMIIPFAPLDYVTGASSYIPITVTVAPMSAEYNGLRLAGHQGLPTMLTPGSTQFLTSDDFQSPSAMPQFDVTPEMNIPGQVRNLMEIAEVDSVVPINNLKANLMTMEAYRVQVRSTDEMGGQIFGFPLQPGASSVLQRTLLGEILNYYTHWSGSLKLTFVFCGSAMATGKFLLAYSPPGAGAPDSRKNAMLGTHVIWDVGLQSSCVLCVPWISQTHYRYVVDDKYTASGFISCWYQTNVIVPAEAQKSCYIMCFVSACNDFSVRMLRDTQFIKQTNFYQGPTEESVERAMGRVADTIARGPSNSEQIPALTAVETGHTSQVDPSDTMQTRHVHNYHSRSESSIENFLCRSACVIYIKYSSAESNNLKRYAEWVINTRQVAQLRRKMEMFTYIRCDMELTFVITSHQEMSTATNSDVPVQTHQIMYVPPGGPVPTSVNDYVWQTSTNPSIFWTEGNAPPRMSIPFMSIGNAYTMFYDGWSNFSRDGIYGYNSLNNMGTIYARHVNDSSPGGLTSTIRIYFKPKHVKAYVPRPPRLCQYKKAKSVNFDVEAVTAERASLITTGPYGHQSGAVYVGNYKVVNRHLATHVDWQNCVWEDYNRDLLVSTTTAHGCDTIARCQCTTGVYFCASKSKHYPVSFEGPGLVEVQESEYYPKRYQSHVLLATGFSEPGDCGGILRCEHGVIGLVTMGGEGVVGFADVRDLLWLEDDAMEQGVKDYVEQLGNAFGSGFTNQICEQVNLLKESLVGQDSILEKSLKALVKIISALVIVVRNHDDLITVTATLALIGCTSSPWRWLKHKVSQYYGIPMAERQNNGWLKKFTEMTNACKGMEWIAVKIQKFIEWLKVKILPEVKEKHEFLSRLKQLPLLESQIATIEQSAPSQSDQEQLFSNVQYFAHYCRKYAPLYAAEAKRVFSLEKKMSNYIQFKSKCRIEPVCLLLHGSPGAGKSVATNLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRKTQVRYSLDMLVTEMFREYNHRHSVGATLEALFQGPPVYREIKISVTPETPPPPVIADLLKSVDRQAIREYCKEKGWLVPEIDSILQIEKHVSRAFICLQALTTFVSVAGIIYIIYKLFAGFQGAYTGMPNQKPKVPTLRQAKVQGPAFEFAVAMMKRNSSTVKTEYGEFTMLGIYDRWAVLPRHAKPGPTILMNDQEVGVLDAKELIDRDGTNLELTLLKLNRNEKFRDIRGFLAKEEVEVNEAVLAINTSKFPNMYIPVGRVTDYGFLNLGGTPTKRMLMYNFPTRAGQCGGVLMSTGKVLGIHVGGNGHQGFSAGLLKHYFNDEQGEIEFIESSKDAGFPVINTPSRTKLEPSVFHHVFEGNKEPAVLRNGDPRLKVNFEEAIFFKYIGNVNTHVDEYMLEAVDHYAGQLATLDINTEPMKLEDAVYGTEGLEALDLTTSAGYPYVALGIKKRDILSKKTKDLTKLKECMDKYGLNLPMVTYVKDELRSAEKVAKGKSRLIEASSLNDSVAMRQTFGNLYKAFHLNPGIVTGSAVGCDPDVFWSKIPVMLDGHLIAFDYSGYDASLSPVWFACLKLLLEKLGYTHKETNYIDYLCNSHHLYRDKHYFVRGGMPSGCSGTSIFNSMINNIIIRTLMLKVYKGIDLDQFRMIAYGDDVIASYPWPIDASLLAEAGKDYGLIMTPADKGECFNEVTWTNVTFLKRYFRADEQYPFLVHPVMPMKDIHESIRWTKDPKNTQDHVRSLCLLAWHNGEHEYEEFIQKIRSVPVGRCLTLPAFSTLRRKWLDSF

SEQ ID NO: 17 - DNA, 20 bp, Artificial Sequence
  Synthetic forward primer used for CBV4 amplification
ttaaaacagcctgtgggttg

SEQ ID NO: 18 - DNA, 22 bp, Artificial Sequence
  Synthetic forward primer used for CBV4 amplification
cggctaatcctaactgcggagc

SEQ ID NO: 19 - DNA, 22 bp, Artificial Sequence
  Synthetic forward primer used for CBV4 amplification
gtagtcctccggcccctgaatg

SEQ ID NO: 20 - DNA, 22 bp, Artificial Sequence
  Synthetic forward primer used for CBV4 amplification
tggctgcttatggtgacaattg

SEQ ID NO: 21 - DNA, 22 bp, Artificial Sequence
  Synthetic forward primer used for CBV4 amplification
tatagctattggattggccatc

SEQ ID NO: 22 - DNA, 20 bp, Artificial Sequence
  Synthetic forward primer used for CBV4 amplification
atgcaaacaagacatgtgca

SEQ ID NO: 23 - DNA, 22 bp, Artificial Sequence
  Synthetic forward primer used for CBV4 amplification
caaataatgtacgtsccacctg

SEQ ID NO: 24 - DNA, 19 bp, Artificial Sequence
  Synthetic forward primer used for CBV4 amplification
tgtggttggargatgaygc

SEQ ID NO: 25 - DNA, 17 bp, Artificial Sequence
  Synthetic forward primer used for CBV4 amplification
atgagcaattacataca

SEQ ID NO: 26 - DNA, 21 bp, Artificial Sequence
  Synthetic forward primer used for CBV4 amplification
tcaagtccaaatgccgtattg

SEQ ID NO: 27 - DNA, 21 bp, Artificial Sequence
  Synthetic forward primer used for CBV4 amplification
atgccgtattgaacctgtatg

SEQ ID NO: 28 - DNA, 20 bp, Artificial Sequence
  Synthetic forward primer used for CBV4 amplification
ggyatctatgacaggtgggc

SEQ ID NO: 29 - DNA, 20 bp, Artificial Sequence
  Synthetic forward primer used for CBV4 amplification
atgctmatgtacaacttccc

SEQ ID NO: 30 - DNA, 21 bp, Artificial Sequence
  Synthetic forward primer used for CBV4 amplification
tacagagacaaacactacttt

SEQ ID NO: 31 - DNA, 20 bp, Artificial Sequence
  Synthetic forward primer used for CBV4 amplification
aggtaytttagagcagatga

SEQ ID NO: 32 - DNA, 21 bp, Artificial Sequence
  Synthetic forward primer used for CBV4 amplification
ttttaaattagasacaatttg

SEQ ID NO: 33 - DNA, 19 bp, Artificial Sequence
  Synthetic forward primer used for CBV4 amplification
ttggcttaaccctactgca

SEQ ID NO: 34 - DNA, 24 bp, Artificial Sequence
  Synthetic forward primer used for CBV4 amplification
gtaaattctccgcrttcggtgcgg

SEQ ID NO: 35 - DNA, 22 bp, Artificial Sequence
  Synthetic forward primer used for CBV4 amplification
ttaaaacagcctgtgggttgta

SEQ ID NO: 36 - DNA, 23 bp, Artificial Sequence
  Synthetic forward primer used for CBV4 amplification
tatctgtttgttggtttcgttcc

SEQ ID NO: 37 - DNA, 21 bp, Artificial Sequence
  Synthetic forward primer used for CBV4 amplification
gaagttcccagatgcattgtc

SEQ ID NO: 38 - DNA, 22 bp, Artificial Sequence
  Synthetic forward primer used for CBV4 amplification
atgagcgctgagtacaatggtt

SEQ ID NO: 39 - DNA, 21 bp, Artificial Sequence
  Synthetic forward primer used for CBV4 amplification
gtcatatgggacgttggactg

SEQ ID NO: 40 - DNA, 22 bp, Artificial Sequence
  Synthetic forward primer used for CBV4 amplification
aatcaaacaacctgaagcggta

SEQ ID NO: 41 - DNA, 22 bp, Artificial Sequence
  Synthetic forward primer used for CBV4 amplification
cccgtttgtgtcaatacaagaa

SEQ ID NO: 42 - DNA, 21 bp, Artificial Sequence
  Synthetic forward primer used for CBV4 amplification
acctgttgtggttggaagatg

SEQ ID NO: 43 - DNA, 21 bp, Artificial Sequence
  Synthetic forward primer used for CBV4 amplification
agagtcagattgccaccattg

SEQ ID NO: 44 - DNA, 21 bp, Artificial Sequence
  Synthetic forward primer used for CBV4 amplification
ctctggcaagaaggttccatt

SEQ ID NO: 45 - DNA, 21 bp, Artificial Sequence
  Synthetic forward primer used for CBV4 amplification
gcatatacggggatgcctaac

SEQ ID NO: 46 - DNA, 22 bp, Artificial Sequence
  Synthetic forward primer used for CBV4 amplification
tactttaatgatgagcaggggg

SEQ ID NO: 47 - DNA, 22 bp, Artificial Sequence
  Synthetic forward primer used for CBV4 amplification
ttagatcagcagagaaggtggc

SEQ ID NO: 48 - DNA, 21 bp, Artificial Sequence
  Synthetic forward primer used for CBV4 amplification
gccagcgctcaattccccgra

SEQ ID NO: 49 - DNA, 18 bp, Artificial Sequence
  Synthetic forward primer used for CBV4 amplification
gaccaacagaagagtccg

SEQ ID NO: 50 - DNA, 18 bp, Artificial Sequence
  Synthetic forward primer used for CBV4 amplification
tgaagcggtatgcggagt

SEQ ID NO: 51 - DNA, 18 bp, Artificial Sequence
  Synthetic forward primer used for CBV4 amplification
gtgatcaccagccatcag

SEQ ID NO: 52 - DNA, 18 bp, Artificial Sequence
  Synthetic forward primer used for CBV4 amplification
cactacccagttagcttt

SEQ ID NO: 53 - DNA, 18 bp, Artificial Sequence
  Synthetic forward primer used for CBV4 amplification
gctacactcgcccttatt

SEQ ID NO: 54 - DNA, 18 bp, Artificial Sequence
  Synthetic forward primer used for CBV4 amplification
ctcaagtgaggtactccc

SEQ ID NO: 55 - DNA, 18 bp, Artificial Sequence
  Synthetic forward primer used for CBV4 amplification
cccaacatgtacatcccc

SEQ ID NO: 56 - DNA, 18 bp, Artificial Sequence
  Synthetic forward primer used for CBV4 amplification
agcgtcttccatcacgtc

SEQ ID NO: 57 - DNA, 22 bp, Artificial Sequence
  Synthetic reverse primer used for CBV4 amplification
gctccgcagttaggattagccg

SEQ ID NO: 58 - DNA, 22 bp, Artificial Sequence
  Synthetic reverse primer used for CBV4 amplification
cattcaggggccggaggactac

SEQ ID NO: 59 - DNA, 22 bp, Artificial Sequence
  Synthetic reverse primer used for CBV4 amplification
caattgtcaccataagcagcca

SEQ ID NO: 60 - DNA, 22 bp, Artificial Sequence
  Synthetic reverse primer used for CBV4 amplification
gatggccaatccaatagctata

SEQ ID NO: 61 - DNA, 20 bp, Artificial Sequence
  Synthetic reverse primer used for CBV4 amplification
tgcacatgtcttgtttgcat

SEQ ID NO: 62 - DNA, 22 bp, Artificial Sequence
  Synthetic reverse primer used for CBV4 amplification
caggtggsacgtacattatttg

SEQ ID NO: 63 - DNA, 19 bp, Artificial Sequence
  Synthetic reverse primer used for CBV4 amplification
gcrtcatcytccaaccaca

SEQ ID NO: 64 - DNA, 17 bp, Artificial Sequence
  Synthetic reverse primer used for CBV4 amplification
tgtatgtaattgctcat

SEQ ID NO: 65 - DNA, 21 bp, Artificial Sequence
  Synthetic reverse primer used for CBV4 amplification
caatacggcatttggacttga

SEQ ID NO: 66 - DNA, 21 bp, Artificial Sequence
  Synthetic reverse primer used for CBV4 amplification
catacaggttcaatacggcat

SEQ ID NO: 67 - DNA, 20 bp, Artificial Sequence
  Synthetic reverse primer used for CBV4 amplification
gcccacctgtcatagatrcc

SEQ ID NO: 68 - DNA, 20 bp, Artificial Sequence
  Synthetic reverse primer used for CBV4 amplification
gggaagttgtacatkagcat

SEQ ID NO: 69 - DNA, 21 bp, Artificial Sequence
  Synthetic reverse primer used for CBV4 amplification
aaagtagtgtttgtctctgta

SEQ ID NO: 70 - DNA, 20 bp, Artificial Sequence
  Synthetic reverse primer used for CBV4 amplification
tcatctgctctaaartacct

SEQ ID NO: 71 - DNA, 21 bp, Artificial Sequence
  Synthetic reverse primer used for CBV4 amplification
caaattgtstctaatttaaaa

SEQ ID NO: 72 - DNA, 19 bp, Artificial Sequence
  Synthetic reverse primer used for CBV4 amplification
tgcagtagggttaagccaa

SEQ ID NO: 73 - DNA, 24 bp, Artificial Sequence
  Synthetic reverse primer used for CBV4 amplification
ccgcaccgaaygcggagaatttac

SEQ ID NO: 74 - DNA, 22 bp, Artificial Sequence
  Synthetic reverse primer used for CBV4 amplification
tcttttgtgttgacacctgtgc

SEQ ID NO: 75 - DNA, 21 bp, Artificial Sequence
  Synthetic reverse primer used for CBV4 amplification
gttgcattgcacatgaattgt

SEQ ID NO: 76 - DNA, 22 bp, Artificial Sequence
  Synthetic reverse primer used for CBV4 amplification
ggtgattgaaaatcatctgacg

SEQ ID NO: 77 - DNA, 22 bp, Artificial Sequence
  Synthetic reverse primer used for CBV4 amplification
tgaaaccactagccgtgtactt

SEQ ID NO: 78 - DNA, 21 bp, Artificial Sequence
  Synthetic reverse primer used for CBV4 amplification
ccatgtcgcaccgaatataag

SEQ ID NO: 79 - DNA, 22 bp, Artificial Sequence
  Synthetic reverse primer used for CBV4 amplification
ataggggcctgtggttatcaag

SEQ ID NO: 80 - DNA, 22 bp, Artificial Sequence
  Synthetic reverse primer used for CBV4 amplification
ctcgcatatctggttggtgaat

SEQ ID NO: 81 - DNA, 23 bp, Artificial Sequence
  Synthetic reverse primer used for CBV4 amplification
tactttctgcaatagtgagcgaa

SEQ ID NO: 82 - DNA, 22 bp, Artificial Sequence
  Synthetic reverse primer used for CBV4 amplification
tcatcacacgtcttgactgaca

SEQ ID NO: 83 - DNA, 22 bp, Artificial Sequence
  Synthetic reverse primer used for CBV4 amplification
ctggagttcctcttcatcatgg

SEQ ID NO: 84 - DNA, 22 bp, Artificial Sequence
  Synthetic reverse primer used for CBV4 amplification
ggaagacgcttggttctagctt

SEQ ID NO: 85 - DNA, 22 bp, Artificial Sequence
  Synthetic reverse primer used for CBV4 amplification
caaattaccaaatgtttgcctc

SEQ ID NO: 86 - DNA, 21 bp, Artificial Sequence
  Synthetic reverse primer used for CBV4 amplification
gcggagaatttacccctactg

SEQ ID NO: 87 - DNA, 24 bp, Artificial Sequence
  Synthetic reverse primer used for CBV4 amplification
gagtctcaatatgaaataaagagt

SEQ ID NO: 88 - DNA, 18 bp, Artificial Sequence
  Synthetic reverse primer used for CBV4 amplification
ggtgagggtatatagtca

SEQ ID NO: 89 - DNA, 18 bp, Artificial Sequence
  Synthetic reverse primer used for CBV4 amplification
cctccatcgtcatcagat

SEQ ID NO: 90 - DNA, 19 bp, Artificial Sequence
  Synthetic reverse primer used for CBV4 amplification
gcgtgtccctcaacatgcg

SEQ ID NO: 91 - DNA, 18 bp, Artificial Sequence
  Synthetic reverse primer used for CBV4 amplification
actccgcataccgcttca

SEQ ID NO: 92 - DNA, 18 bp, Artificial Sequence
  Synthetic reverse primer used for CBV4 amplification
ccacacgtagtcgttgac

SEQ ID NO: 93 - DNA, 18 bp, Artificial Sequence
  Synthetic reverse primer used for CBV4 amplification
aaggagtacactgaactg

SEQ ID NO: 94 - DNA, 18 bp, Artificial Sequence
  Synthetic reverse primer used for CBV4 amplification
agtagtgtcagctccaga

SEQ ID NO: 95 - DNA, 26 bp, Artificial Sequence
  Synthetic reverse primer used for CBV4 amplification
ttttttttttttttttttttccgcac